(19) United States
(12) Patent Application Publication
(10) Pub. No.: US 2024/0252986 A1
(43) Pub. Date: Aug. 1, 2024

(54) System for Performing Cleavage, Deprotection, Ultrafiltration, and Diafiltration Operations

(71) Applicant: Asahi Kasei Bioprocess America, Inc, Glenview, IL (US)

(72) Inventors: Cory Hinz, Glenview, IL (US); George Flores, Glenview, IL (US); Brian Crawford, Glenview, IL (US)

(21) Appl. No.: 18/431,574

(22) Filed: Feb. 2, 2024

Related U.S. Application Data
(63) Continuation of application No. 18/394,774, filed on Dec. 22, 2023, now abandoned.
(60) Provisional application No. 63/435,156, filed on Dec. 23, 2022.

(51) Int. Cl. B01D 61/14; B01J 20/283; B01J 20/285; B01J 20/34; C07H 21/00

(52) U.S. Cl. CPC B01D 61/14; B01J 20/283; B01J 20/285; B01J 20/3483; C07H 21/00

(57) ABSTRACT

Provided herein are common systems for performing cleavage, deprotection, ultrafiltration, and diafiltration operations for producing an oligo product. Also provided are methods of synthesizing an oligo product with a common system as disclosed herein, and oligonucleotides synthesized by the methods disclosed herein.

### CROSS-REFERENCE TO RELATED APPLICATIONS

This is a continuation of U.S. patent application Ser. No. 18/394,774, filed Dec. 22, 2023, which claims benefit under 35 U.S.C. § 119(c) of U.S. Ser. No. 63/435,156, filed Dec. 23, 2022, and the disclosures thereof are hereby incorporated by reference in their entirety.

### FIELD OF THE DISCLOSURE

The present disclosure generally relates to a single system that can be used to perform cleavage, deprotection, ultrafiltration, and diafiltration operations for producing oligonucleotides.

### BACKGROUND

The cleavage, deprotection, ultrafiltration, and diafiltration processes required for oligonucleotide production pose several challenges. First, known systems that perform the cleavage and deprotection processes are very rudimentary in nature (they are typically manually or substantially manually performed) and require auxiliary equipment (e.g., a separate tank). Second, deprotection processes, particularly for RNA production, require the addition of an acid that is exothermic, which increases the temperature in a way that is highly detrimental to the process and is difficult to control. Third, known ultrafiltration and diafiltration systems lack the necessary certification to be operated in the classified hazardous electrical area within which oligo production is conducted (which is so classified because of solvent handling), thereby requiring the product to be transported from the cleavage and deprotection system(s) to a different area within a facility that is safe for the ultrafiltration and diafiltration processes to be performed. Fourth, known diafiltration systems require a large vessel in order to accommodate dosing of exchange buffer solution, after which the diafiltration operation again reduces the volume.

### BRIEF DESCRIPTION OF THE DRAWINGS

The features of this disclosure which are believed to be novel are set forth with particularity in the appended claims. The present disclosure may be best understood by reference to the following description taken in conjunction with the accompanying drawings, in which like reference numerals identify like elements in the several figures, in which:

FIGS. 1A-1I are a schematic illustration of an example of a common system constructed in accordance with the teachings of the present disclosure, showing the system performing the first part of a cleavage process for a product;

FIGS. 2A-2I are similar to FIGS. 1A-1I, but show the system performing the second part of the cleavage process for the product;

FIGS. 3A-3I are similar to FIGS. 1A-1I, but show the same system performing a first part of a deprotection process for the product;

FIGS. 4A-4I are similar to FIGS. 1A-1I, but show the system performing a second part of the deprotection process for the product;

FIGS. 5A-5I are similar to FIGS. 1A-1I, but show the system performing a third part of the deprotection process for the product;

FIGS. 6A-6I are similar to FIGS. 1A-1I, but show the same system performing a first part of an ultrafiltration process for the product;

FIGS. 7A-7I are similar to FIGS. 1A-1I, but show the system performing a second part of the ultrafiltration process for the product;

FIGS. 8A-8I are similar to FIGS. 1A-1I, but show the same system performing a diafiltration process for the product; and

FIGS. 9A-9I are similar to FIGS. 1A-1I, but show the system performing a collection process for the product.

### DETAILED DESCRIPTION

The present disclosure is directed to a common system for automatically (or substantially automatically), efficiently, and safely performing each of the cleavage, deprotection, ultrafiltration, and diafiltration operations necessary to produce oligonucleotides. By utilizing a common (or single) system for all of these operations, the process for producing oligonucleotides is more efficient, less risky, and requires significantly less equipment than the process performed utilizing known systems. Specifically, the single system utilizes the same pumps, the same vessel, and the same processing equipment for all of the cleavage, deprotection, ultrafiltration, and diafiltration operations, such that fewer pumps (e.g., three instead of four), fewer vessels (e.g., one instead of two), and fewer processing equipment (e.g., one processor instead of two, three, or four) is/are needed. At the same time, the single system will be operable in hazardous areas (because the system will be certifiable for use in hazardous electrical areas according to the NEC/ATEX/IECEx standards), such that the ultrafiltration and diafiltration operations can be performed in hazardous areas and the product will no longer need to be transported to different locations and different equipment between operations, thereby reducing risk and processing time.

The common system also beneficially offers combined feed and recirculation functionality, recirculation with an-line heat exchanger for optimized temperature control over critical steps, and inline dilution during the diafiltration operation and, more particularly, during diafiltration buffer exchange (instead of utilizing batch-style dosing into a larger, separate tank). The common system also has an optimized design that minimizes the portions of the common system that need to have a high corrosion resistance. For example, the common system is designed so that only the process vessel and the deprotection components (e.g., the deprotection pump and the conduit connecting the deprotection pump to the process vessel) need to be made of a material having a high corrosion resistance (e.g., Hastelloy® or a similar corrosion resistant alloy), whereas the rest of the common system can be made of stainless steel (e.g., 316/316 L stainless steel), which is much cheaper than Hastelloy® and other high corrosion resistant materials.

Also provided herein are methods of synthesizing an oligo product, comprising: performing a solid-phase synthesis step using an oligo column to produce an oligo product; performing a cleavage step to detach the oligo product from a solid support within the oligo column; optionally performing a deprotection step to process the oligo product post solid-phase-synthesis; performing an ultrafiltration step to separate the oligo product from waste products of the solid-phase synthesis; and performing a diafiltration step to carry out one or more buffer exchanges, wherein the solid-phase synthesis step, cleavage step, deprotection step, ultrafiltration step, and diafiltration step are performed using a common system. In some embodiments, the common system is a common system disclosed herein. In some embodiments, the methods comprise performing the deprotection step.

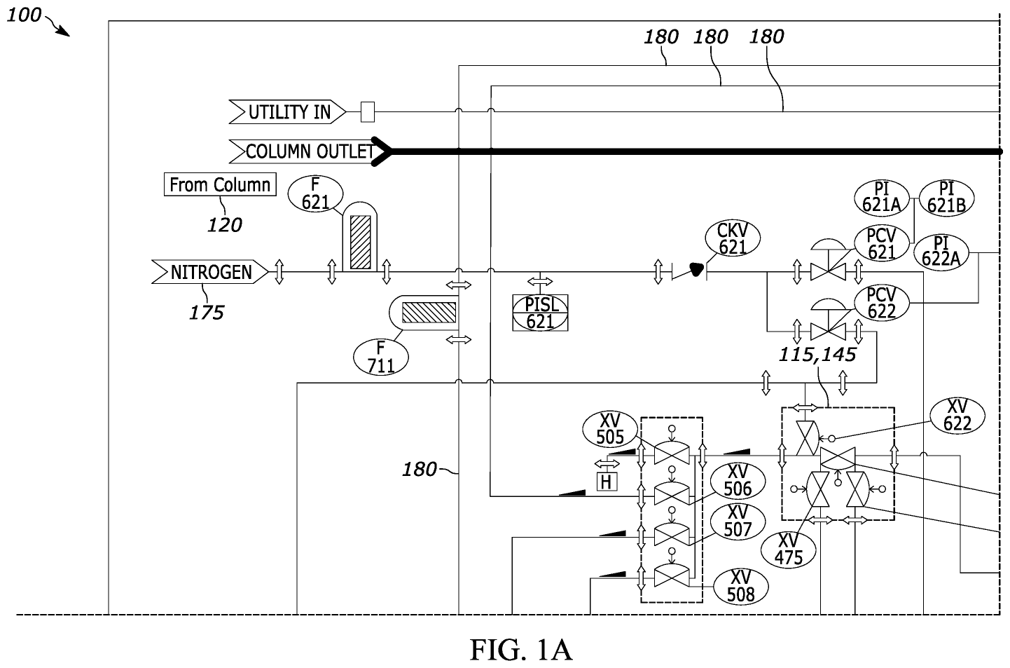

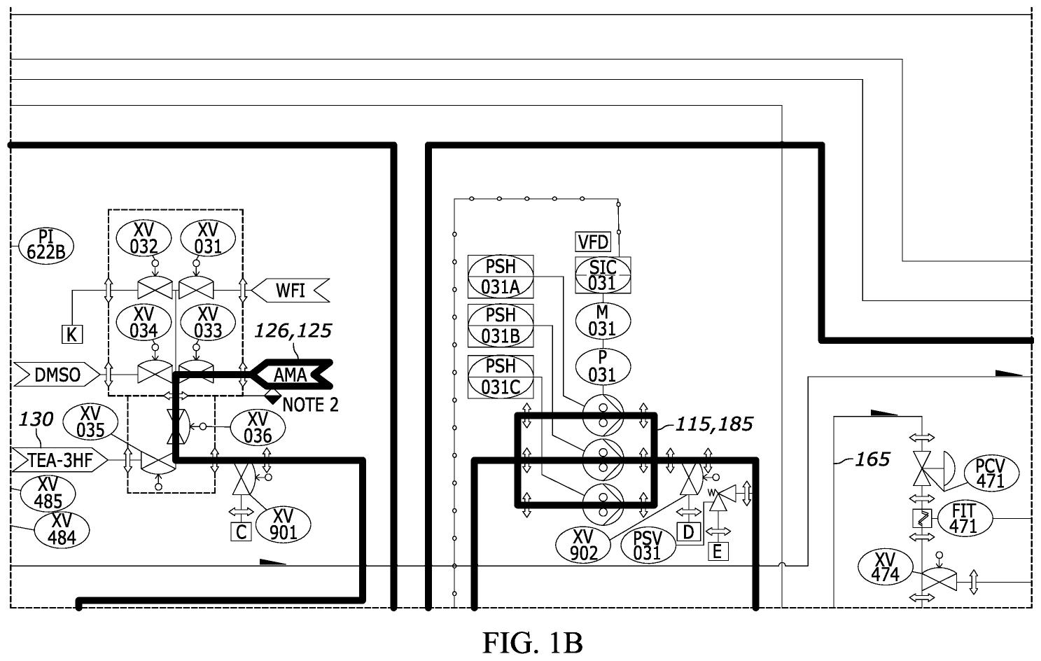

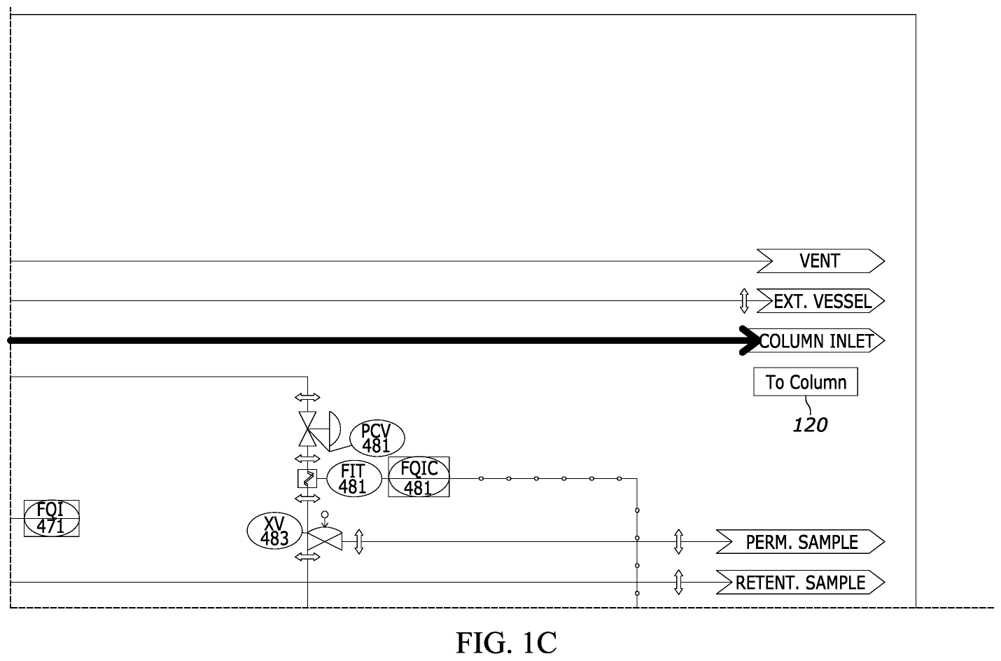

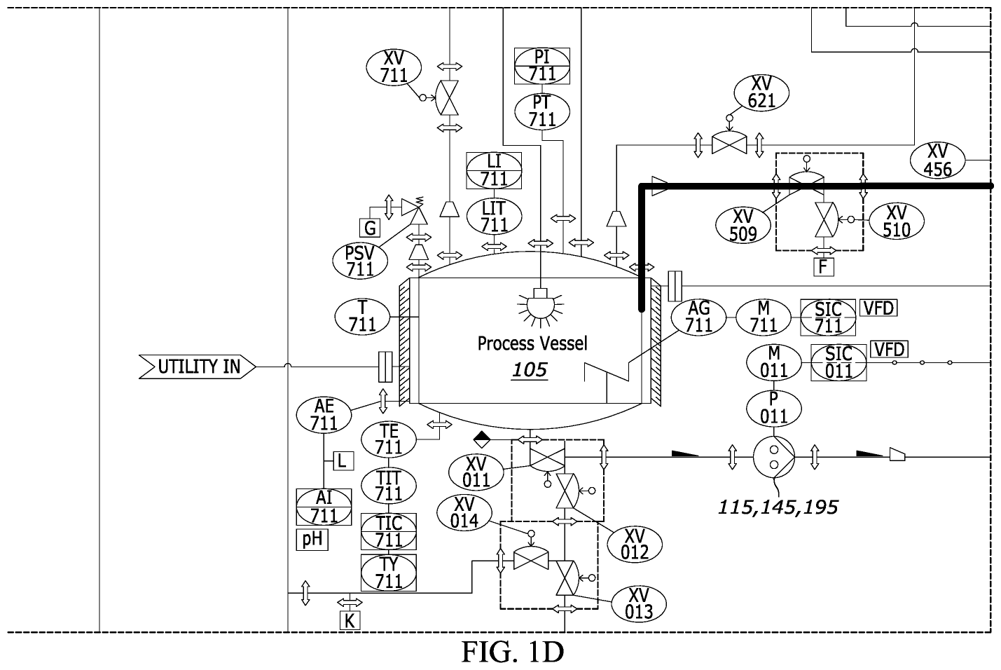

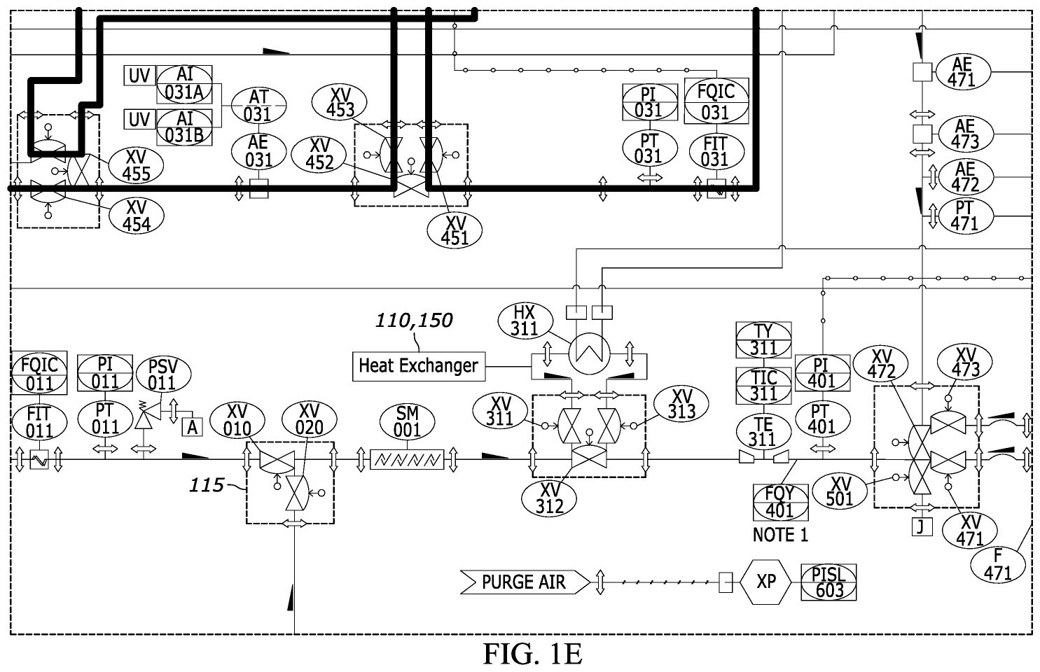

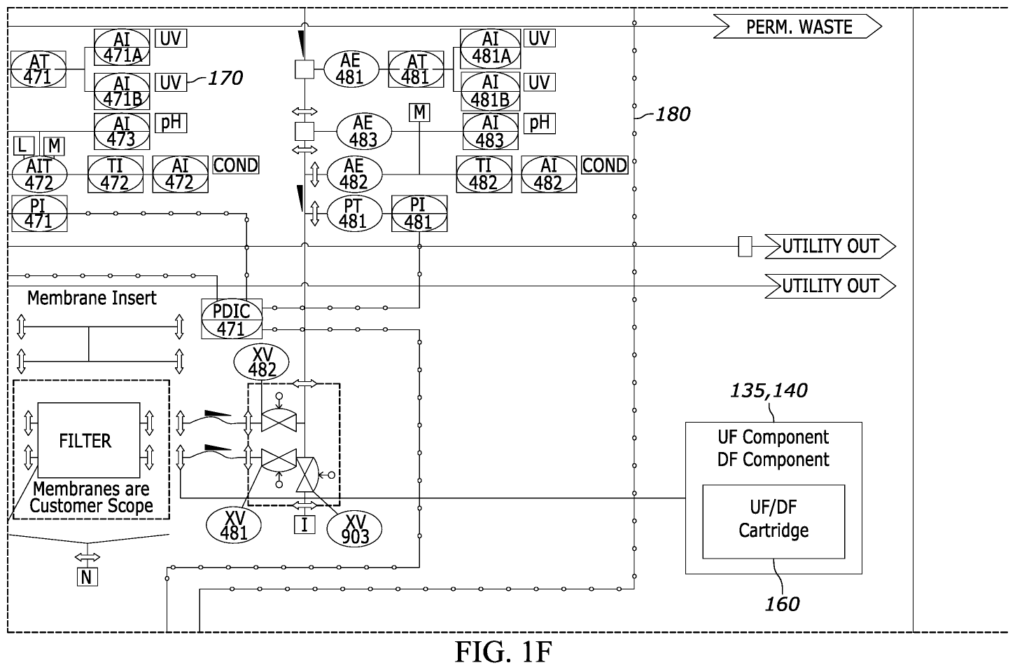

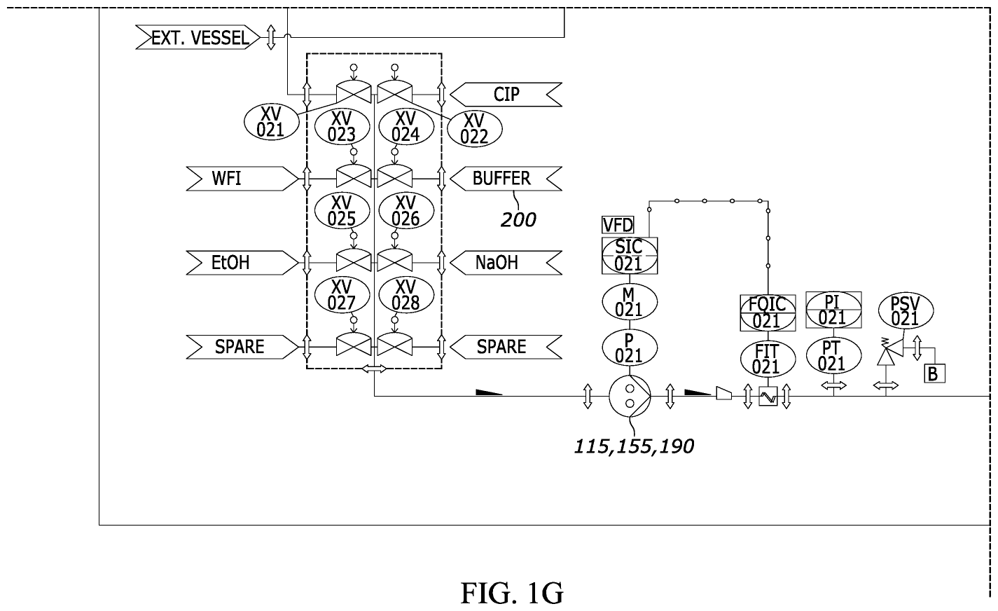

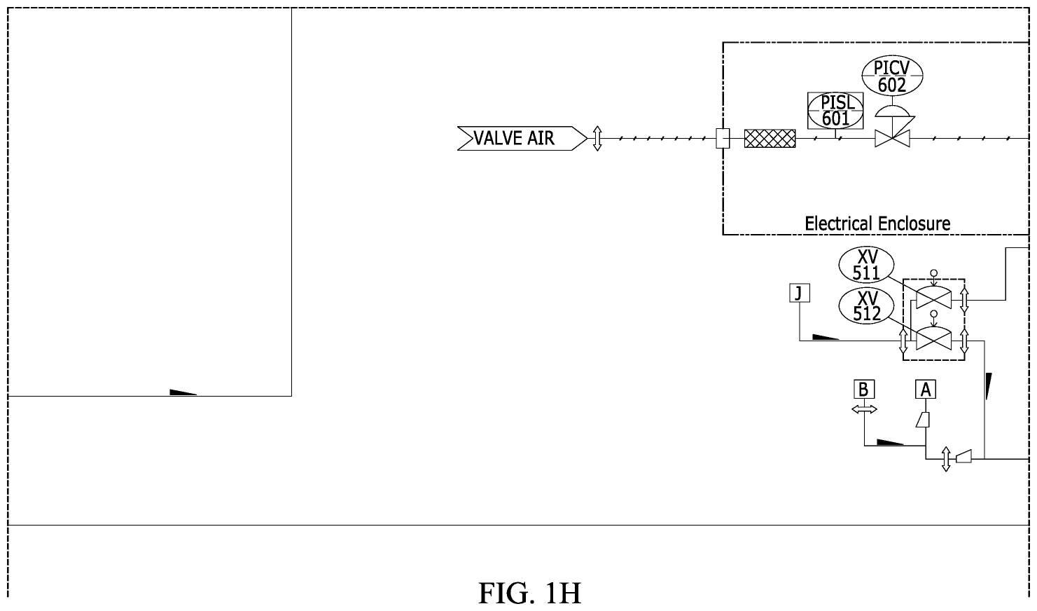

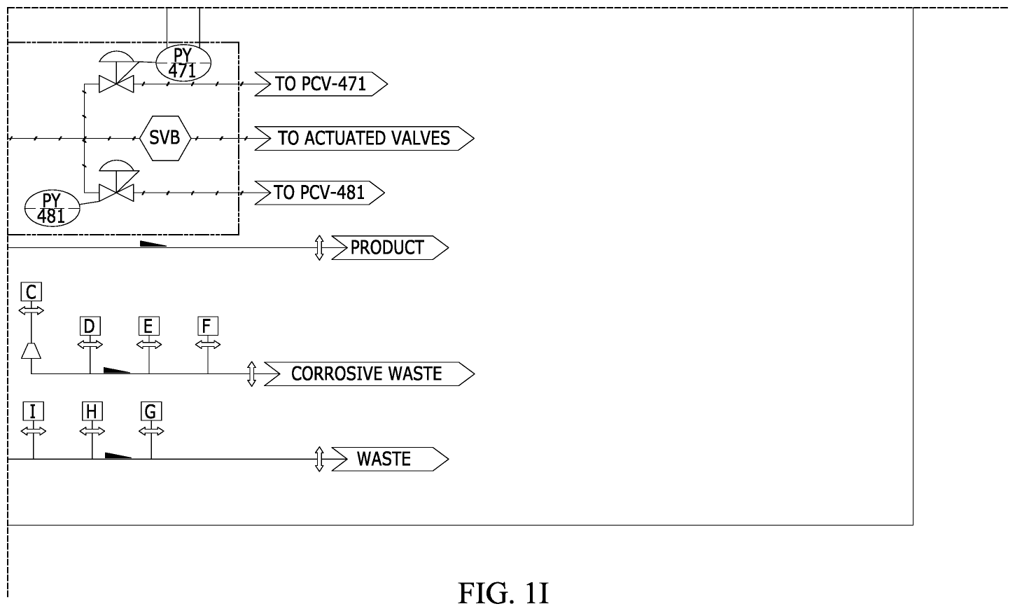

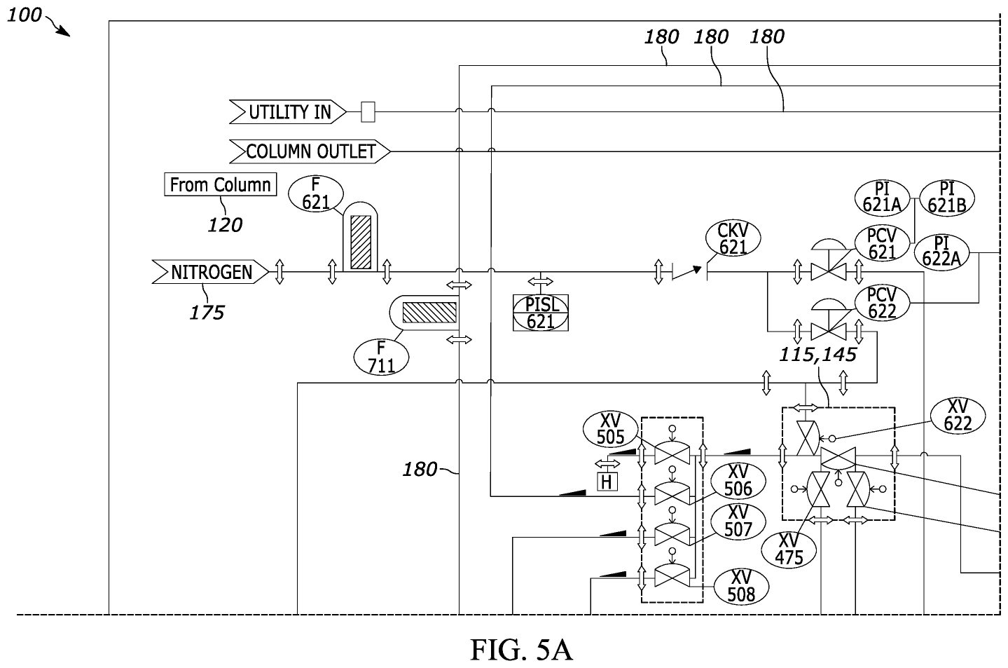

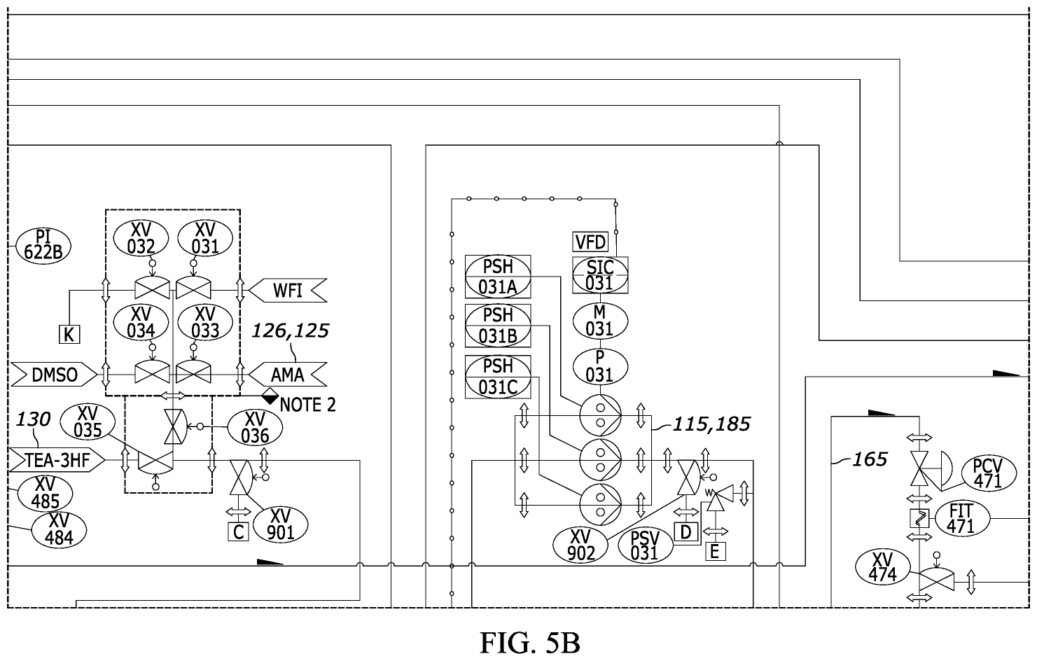

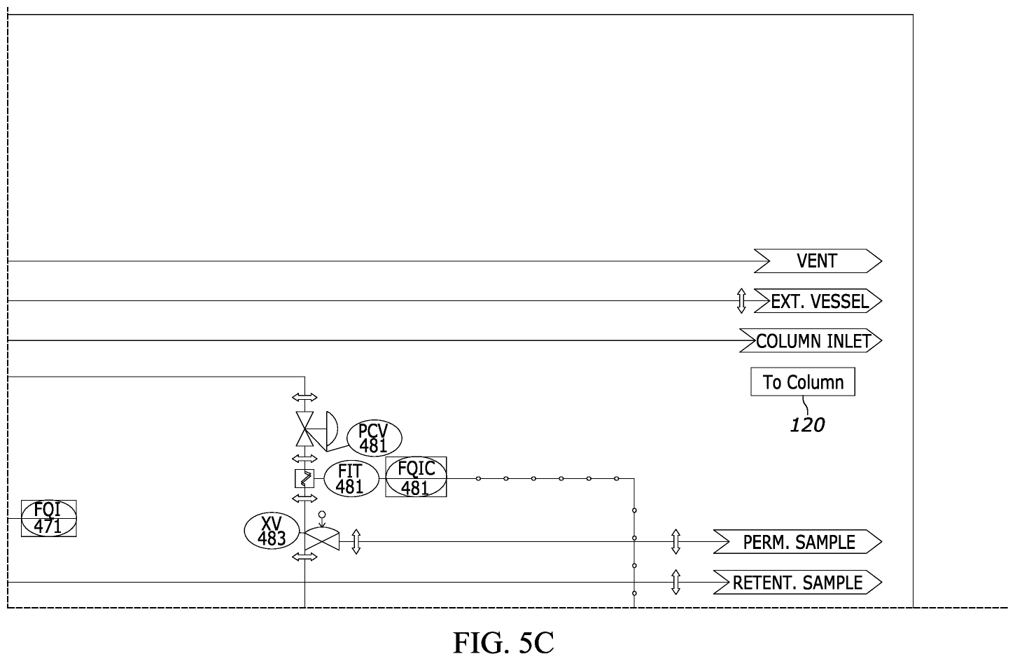

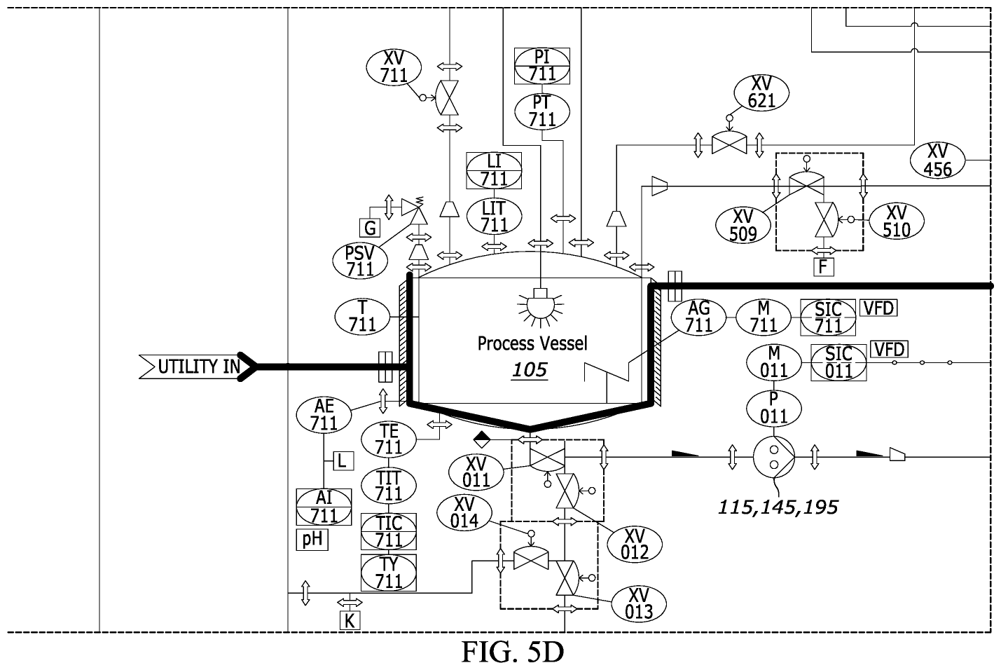

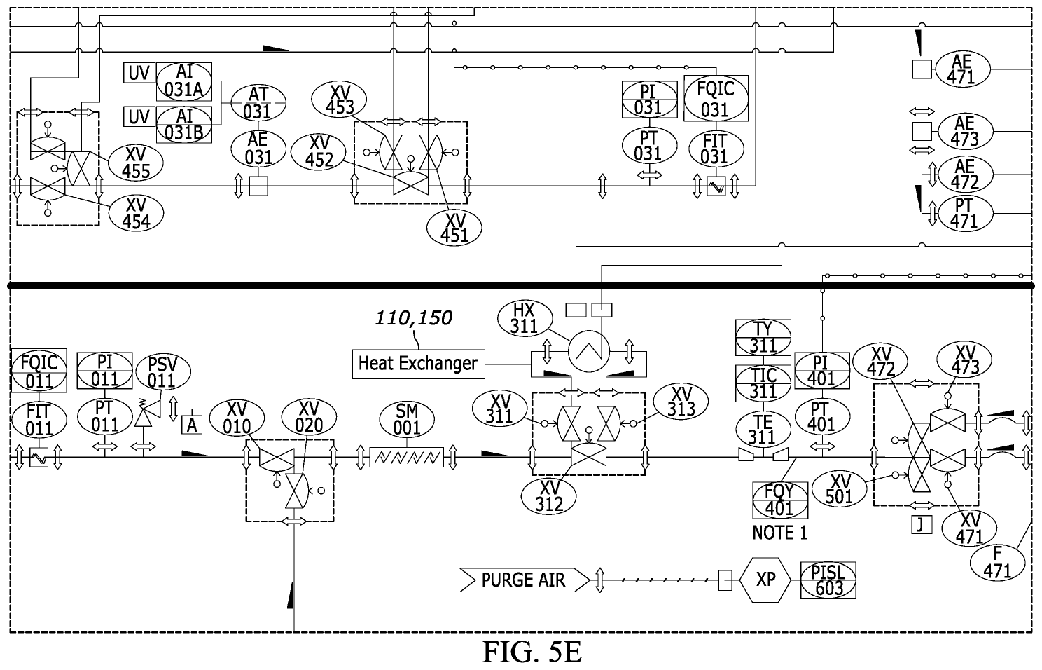

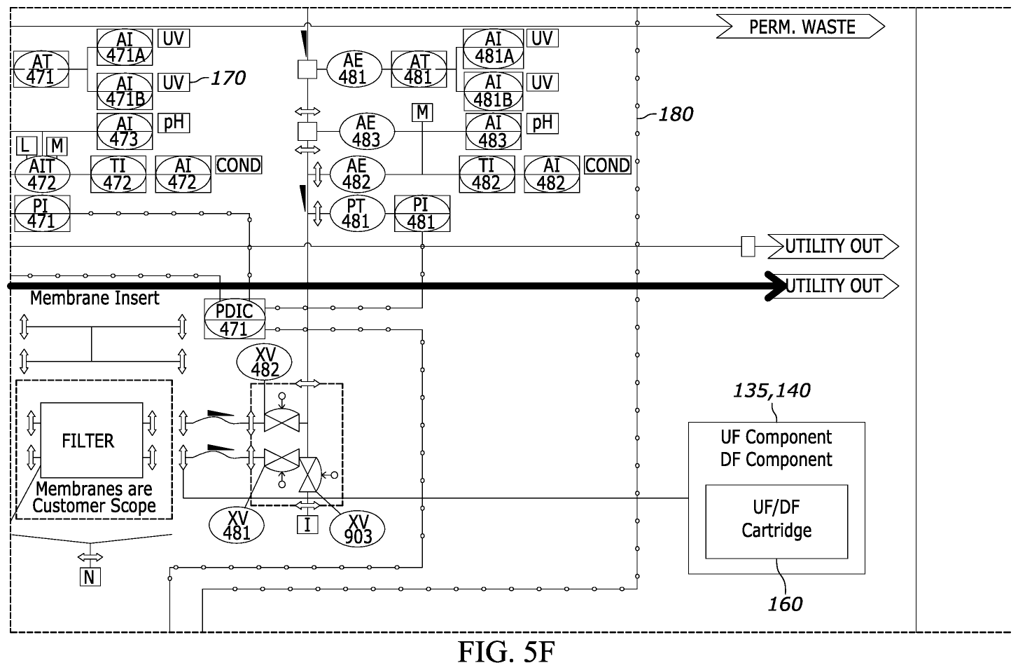

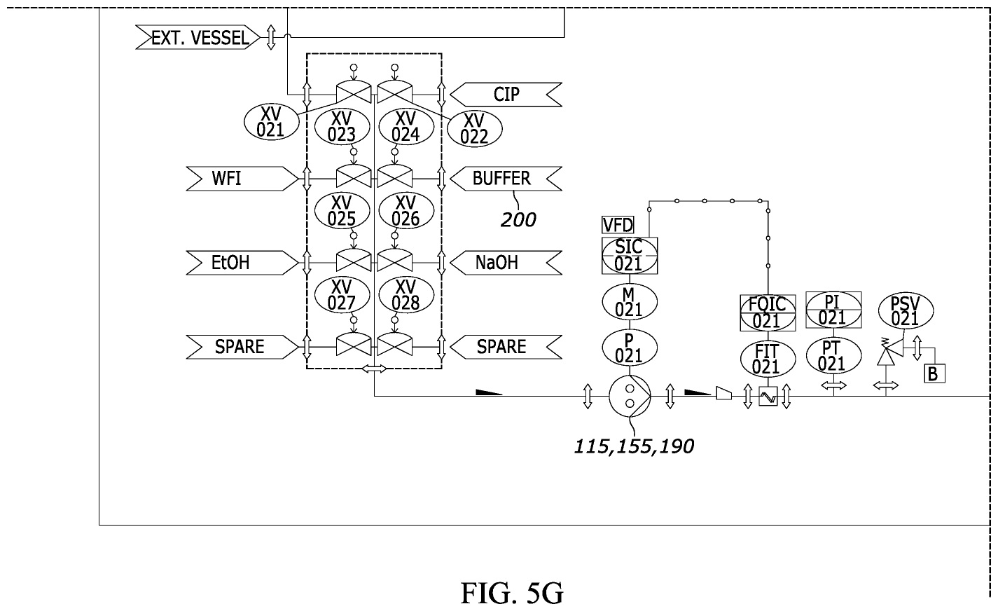

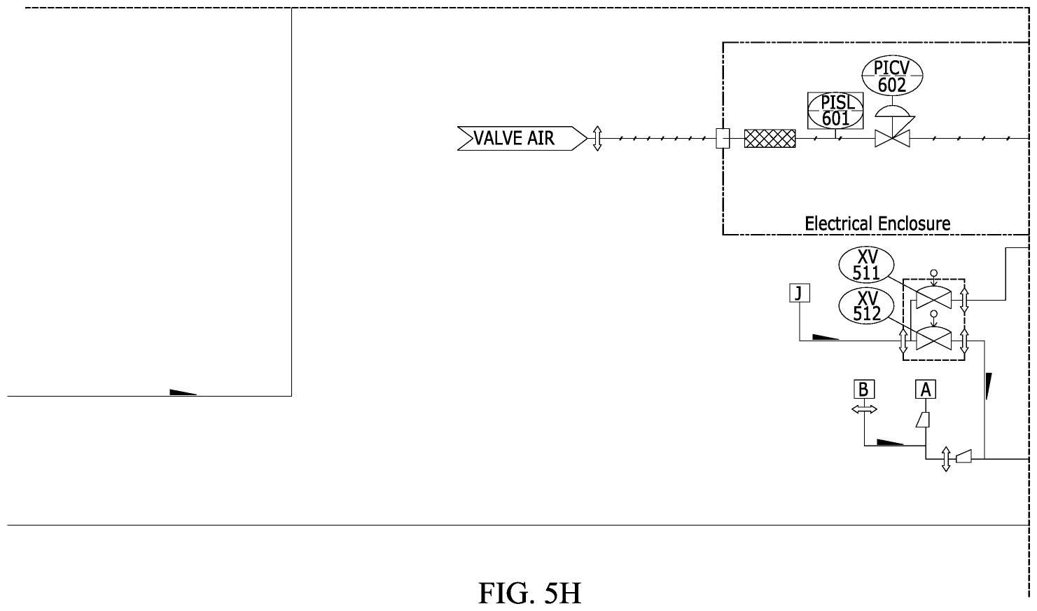

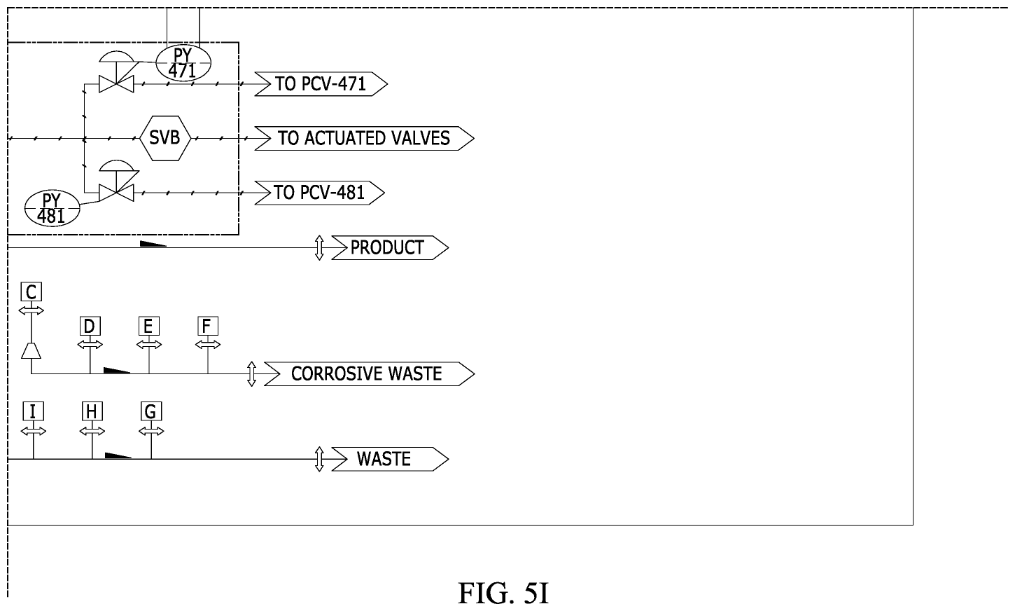

FIGS. 1A-9I illustrate an embodiment of a common system 100 constructed in accordance with the teachings of the present disclosure. Thus, the system can be utilized to perform all of the necessary operations for producing oligonucleotides, namely the cleavage, deprotection, ultrafiltration, and diafiltration operations.

As illustrated in FIGS. 1A-9I, the common system 100 generally employs a single process vessel 105 configured for receiving and/or performing post-synthesis processing of the oligo product; an inline temperature control element 110 paired with the single process vessel 105; one or more pumps 115; optionally, an oligo column 120 configured for solid-phase synthesis of the oligo product; optionally, a cleavage component 125 configured to cleave the oligo product from a solid support within the oligo column; a deprotection component 130 configured for post-synthesis processing of the oligo product; an ultrafiltration component 135 configured for removing waste products of the solid-phase synthesis; and a diafiltration component 140 configured for performing one or more buffer exchanges. The process vessel 105, the one or more pumps 115, the cleavage component 125, the deprotection component 130, the ultrafiltration component 135, and the diafiltration component 140 are connected to each other via one or more conduits 180 (e.g., process piping and/or hoses). The one or more pumps 115 are configured to fluidly connect i) the cleavage component 125 to the oligo column 120; ii) the oligo column to the process vessel 105; iii) the deprotection component 130 to the process vessel; and iv) the ultrafiltration and diafiltration components 135 and 140 to the process vessel and to waste.

In this embodiment, the common system 100 further comprises the oligo column 120 configured for solid-phase synthesis of the oligo product. In some embodiments, the oligo column 120 comprises a controlled pore glass (CPG) or polystyrene (PS) support. In this embodiment, the process vessel 105 is downstream of the oligo column 120. In other embodiments, however, the common system 100 may not include an oligo column 120 of any kind. In the particular embodiment illustrated by FIGS. 1A-9I, the common system 100 comprises the process vessel 105, a recirculation pump 145, an inline heat exchanger 150, a feed pump 155, the oligo column 120, the ultrafiltration component 135, which takes the form of a UF/DF membrane cassette (also called a “UF/DF cartridge”) 160, a recirculation loop 165 with one or more inline analytic devices 170, a nitrogen supply 175, and conduits 180 connecting and extending between the various components.

In this embodiment, the process vessel 105 is jacketed and insulated and is paired with the inline temperature control 110 so as to help control the temperature of the process vessel based on the measured process temperature. In this embodiment, the inline heat control comprises an inline heat exchanger 150. In various embodiments, the inline temperature control element 110 (e.g., inline heat exchanger 150) is paired to the process vessel 105 with a recirculation pump 145 to form a recirculation loop 165. In this embodiment, the process vessel 105 also includes analytic devices 170 as well as a mixer (or agitator) configured to help homogenize the contents of the process vessel during most of the operations. Moreover, because the common system 100 offers recirculation and inline dilution functionality, the process vessel 105 can be smaller and more standardized (and therefore less expensive) than the process vessels typically required for the same batch size. For example, the process vessel 105 can have a working volume of approximately 100 L, which is smaller than conventional process vessels which typically have a working volume of 200 L or more for the same batch size. In some embodiments, the process vessel 105 has a working volume approximately equal to or less than 100 L. As another example, the process vessel 105 can have a basic, cost-effective geometry rather than the expensive conical or stepped vessel configuration sometimes employed.

In this embodiment, the common system 100 comprises a feed pump 155, a deprotection pump 185, and/or a recirculation pump 145. In some embodiments, the systems disclosed herein may not include any additional pumps 115. In one example, the recirculation and feed pumps 145 and 155 will have the appropriate turn-down so as to achieve various operation flow rate targets. In this embodiment, the nitrogen supply 175 helps to enable blanketing of the contents of the process vessel 105, blowdown of the process lines, and integrity testing of the UF/DF membrane cassette 160. In this embodiment, the feed line into the process vessel 105 is designed to de-localize the deprotection acid addition, and, therefore, the corresponding exothermic temperature increase.

### Cleavage

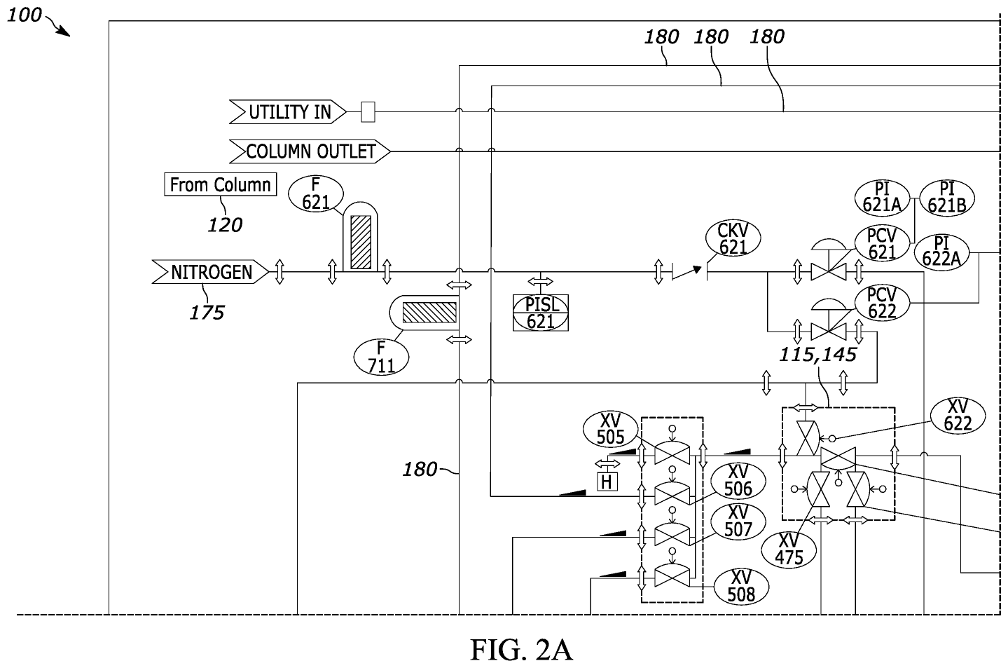

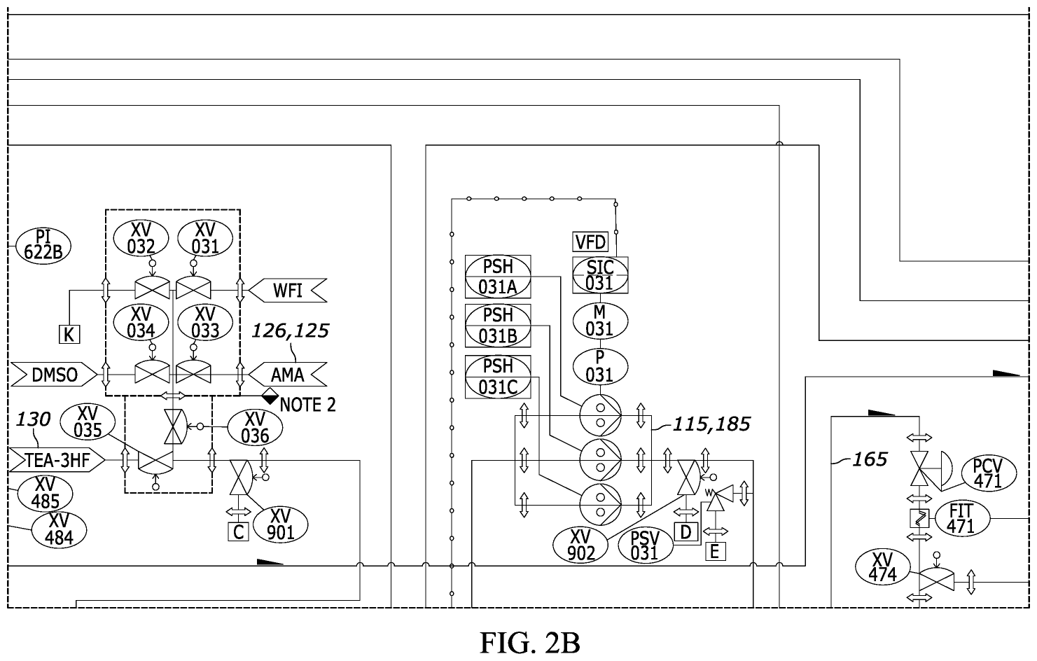

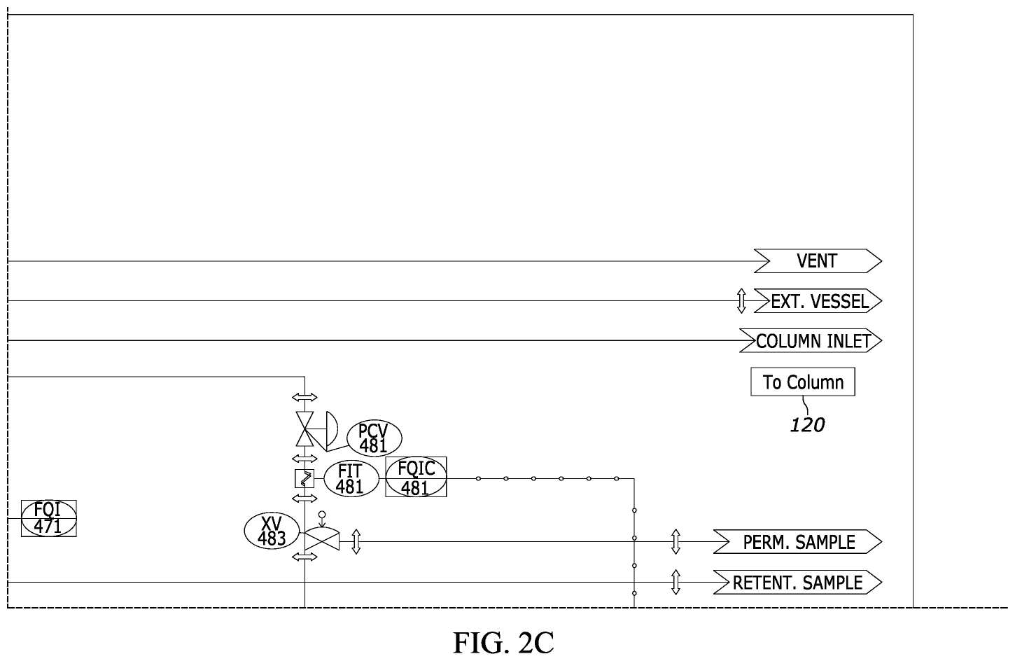

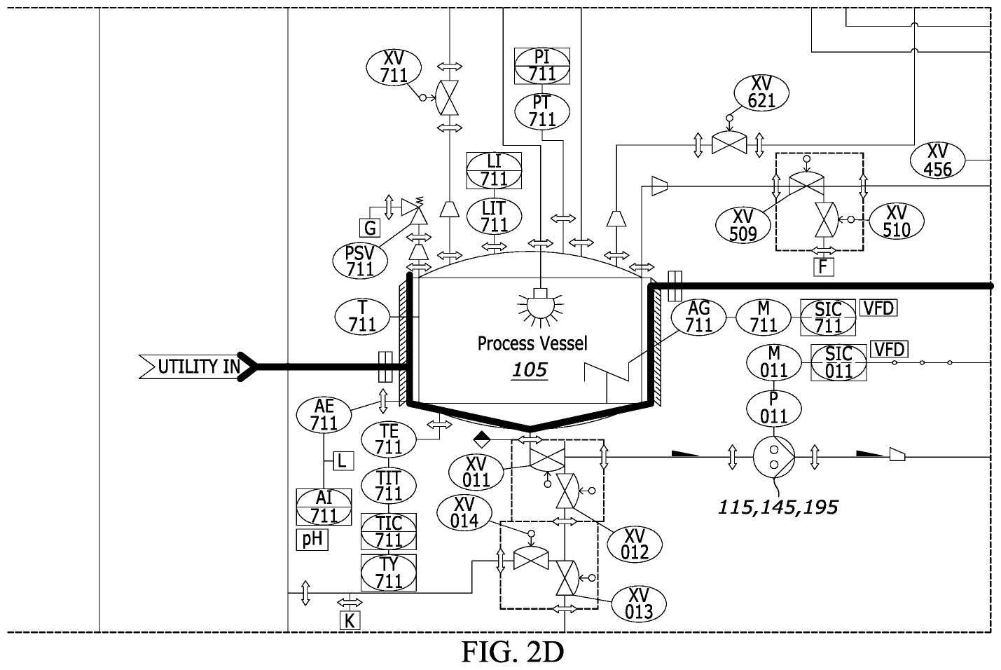

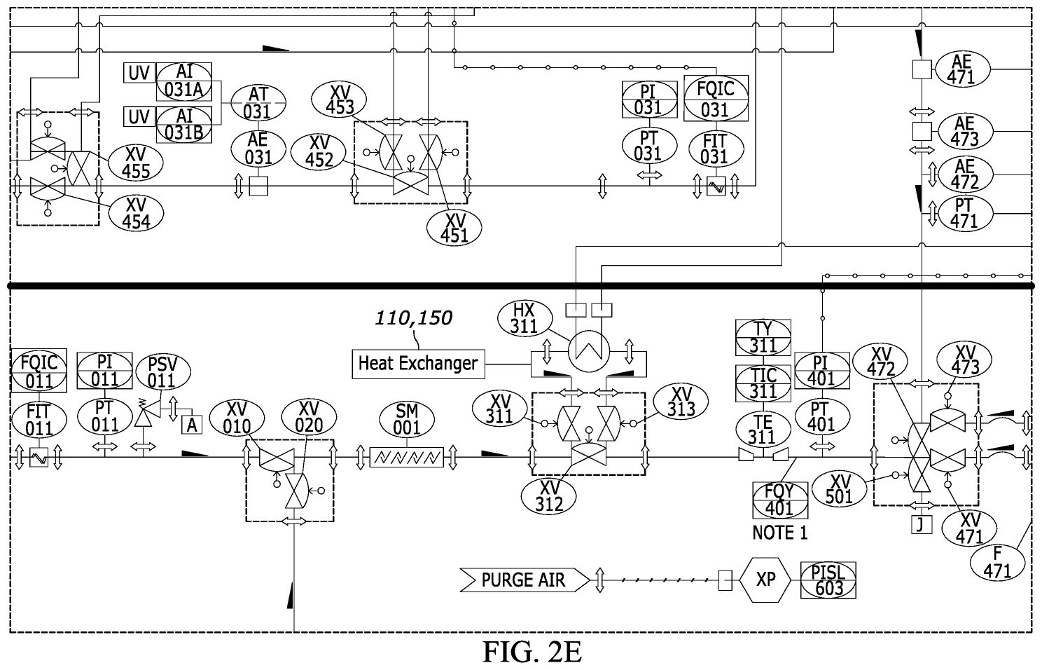

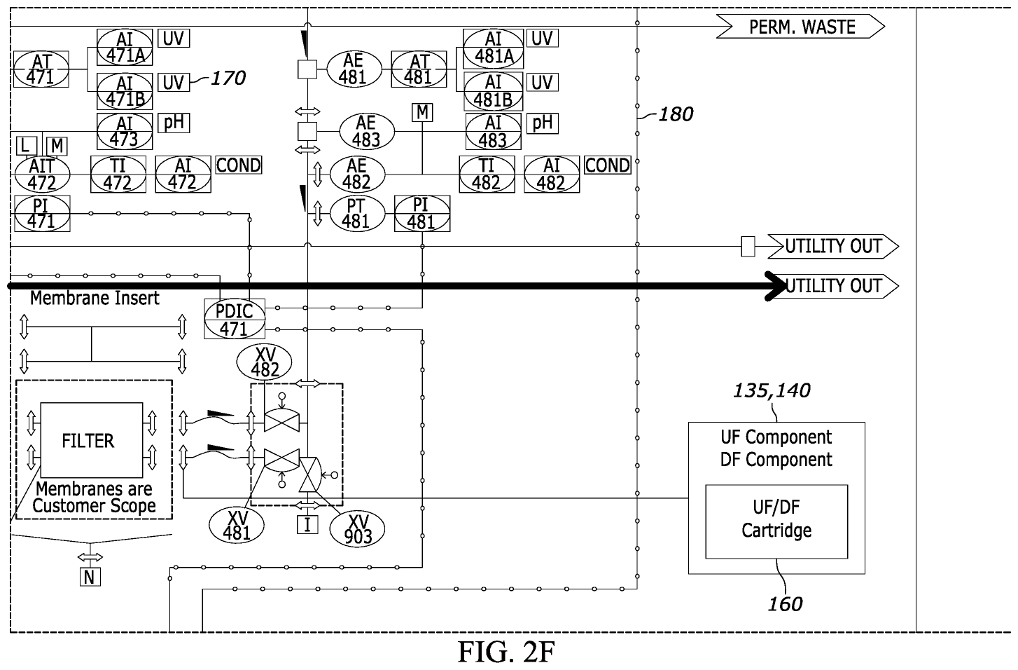

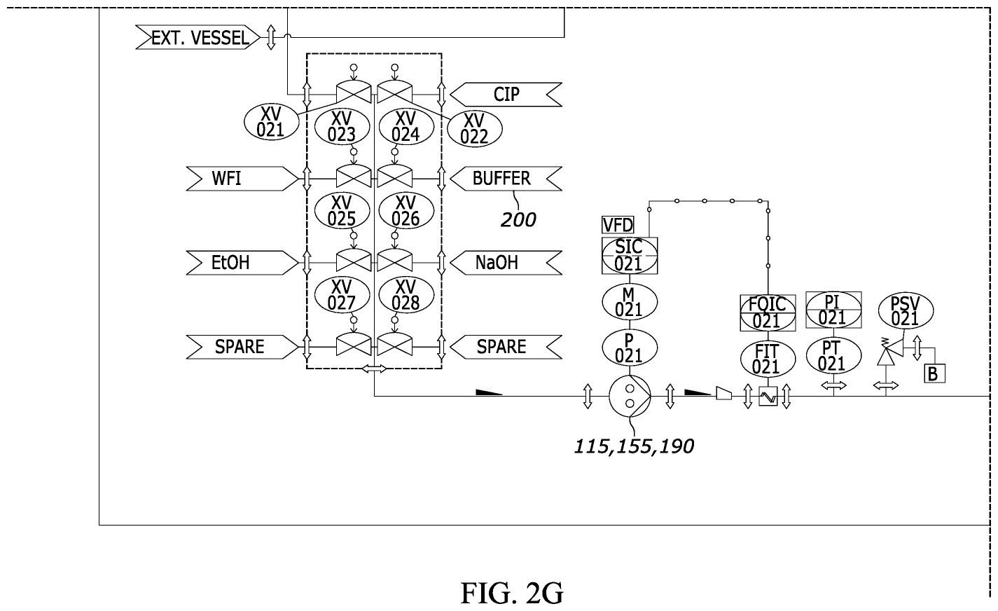

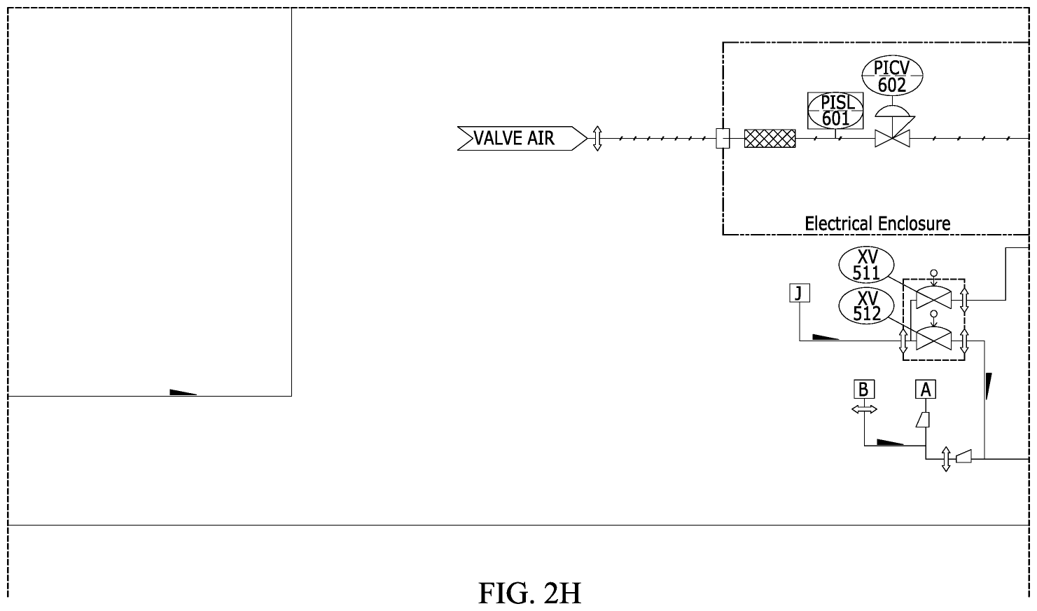

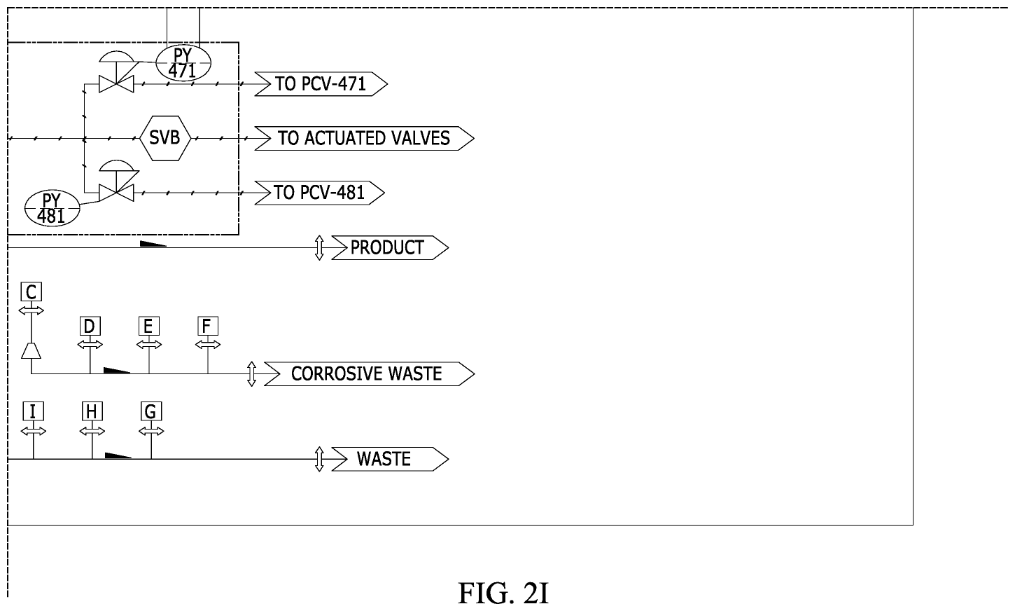

Turning first to FIGS. 1A-1I and 2A-2I, which illustrate how the cleavage step is performed using the common system 100, the cleavage step is generally accomplished by pumping a cleavage component 125 from a supply thereof 126 through the deprotection pump 185 and the attached oligo column 120 to the process vessel 105, resulting in the product being flushed into the process vessel. This step removes the completed DNA or RNA chains from the solid support inside the column 120 and prepares them for further processing. The cleavage step is performed in the system with the cleavage component 125, which in this embodiment is a cleavage solution configured to cleave the oligo product from the solid support (e.g., the oligo column 120) after synthesis.

More particularly, after the oligo product has been synthesized, the oligo product is still attached to the solid support material (e.g., controlled pore glass or polystyrene) inside the column 120. The subsequent process steps ready the molecule for further processing by systematically removing the protective groups that exist on the molecule. The cleavage step cleaves the molecule from the solid support by exposing the column 120 to the cleavage solution, e.g., a solution of, for example, ammonium hydroxide and methylamine (AMA), which breaks the molecule's 3′ bonds. In some embodiments, AMA is directed into the column 120 and held for a specified duration, after which the column contents (including the oligo product) are flushed into the process vessel 105 (which may or may not be empty). In some embodiments, AMA is directed into the column 120 and recirculated (e.g., via the recirculation loop 165) for a specified duration, after which the column contents (including the oligo product) are flushed into the vessel. In some embodiments, the oligo column 120 is exposed to the cleavage solution over less than about 0.5 hours, or over about 1-3 hours. The cleavage solution can be delivered to the column 120 and the column contents can be flushed into the vessel via the deprotection pump 185. Next, the contents of the vessel are heated to a specified temperature for a specified duration, which breaks the molecule's 5′ bonds. This heating can be expedited by recirculating the vessel contents through the inline temperature control element 110 (e.g., heat exchanger).

In some embodiments, the cleavage step can be accomplished either alone or in combination with the deprotection step. In some embodiments, the cleavage step comprises recirculating the cleavage solution through the oligo column 120, or by introducing the cleavage solution into the column and retaining the solution in the column for a specified period of time.

In some embodiments, the cleavage solution comprises AMA. In some embodiments, the cleavage solution is heated to about 25° C. to about 50° ° C. In some embodiments, the cleavage solution is heated to about 40° C. In some embodiments, the column 120 is exposed to the cleavage solution for a period of about 0.5 hours or less, or from about 0.5-3 hours. In some embodiments, the cleavage solution is recirculated through the column 120 for about 0.5 hours or less. In some embodiments, the cleavage solution is retained in the column 120 for about 0.5 hours or less. In some embodiments, the cleavage solution is recirculated through the column 120 for about 1-3 hours. In some embodiments, the cleavage solution is retained in the column 120 for about 1-3 hours. In some embodiments, AMA or an equivalent solution is pumped into the column 120 to release the oligo product (cleavage) using one of the following nonlimiting alternatives: a) AMA heated to about 25-50° C. is recirculated through the column for about 1-3 hours to cleave; AMA heated to about 25-50° C. is left in the column for 1-3 hours to cleave; or AMA is left in the column for less than about 30 minutes to cleave only (with the expectation that heating will be accomplished later in the process vessel).

In some embodiments, the cleavage step is followed by pumping (or flushing) the column contents into the process vessel 105. In some embodiments, the column contents are flushed into the process vessel 105 with the cleavage solution. In some embodiments, the column contents are flushed into the process vessel 105 using a solvent after treatment with the cleavage solution. In some embodiments, the column contents are pumped into the vessel 105 with DMSO or equivalent solution after cleavage using one of the following nonlimiting alternatives: a) AMA is followed by DMSO to flush the oligo product into the process vessel 105 accompanied by AMA and DMSO solutions; or b) AMA is used to flush all the oligo product into the process vessel 105 accompanied by AMA solution, and DMSO solution is then dosed into the process vessel 105 separately. In embodiments where the cleavage step is not performed at elevated temperature (e.g., at about 25-50° C.), the product with the AMA is heated to a specified temperature in the process vessel 105 for a specified period of time (e.g., about 1-3 hours). In one nonlimiting embodiment, the process vessel contents comprising the oligo product, and AMA or AMA/DMSO are heated to about 25-50° C. for about 1-3 hours. In another embodiment, the process vessel contents comprising the oligo product, and AMA or AMA/DMSO are heated to about 40° C. for about 1-3 hours. In some embodiments, heating the contents of the process vessel comprises recirculating the contents of the process vessel 105 through the in-line temperature control element 110, such as an in-line heat exchanger, as illustrated in FIGS. 2A-2I.

### Deprotection

Those skilled in the art of oligonucleotide synthesis (e.g., solid-phase oligonucleotide synthesis) will recognize that a variety of protecting groups are employed during the synthesis in order to avoid unwanted side- or cross-reactions during the synthesis. These protecting groups are typically removed from the final oligonucleotide product. The terms “deprotection”, “deprotecting”, and/or “deprotected” as used herein specifically refer to the removal of a protecting group from the 2′ ribose hydroxyl moiety of an RNA oligonucleotide. A skilled artisan will, however, recognize that the systems and methods disclosed herein can implicitly include other deprotection steps necessary to generate a final DNA or RNA oligonucleotide product, including such deprotection steps not included in the meaning of “deprotection”, “deprotecting”, and/or “deprotected” as used herein (i.e., deprotection steps other than the removal of a protecting group from the 2′ ribose hydroxyl moiety of an RNA oligonucleotide).

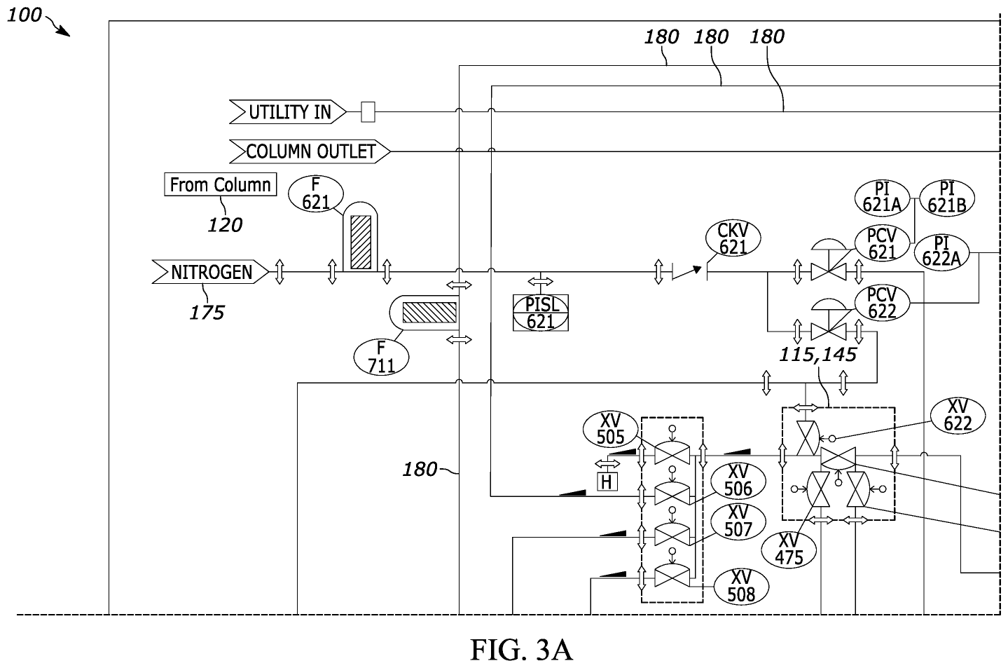

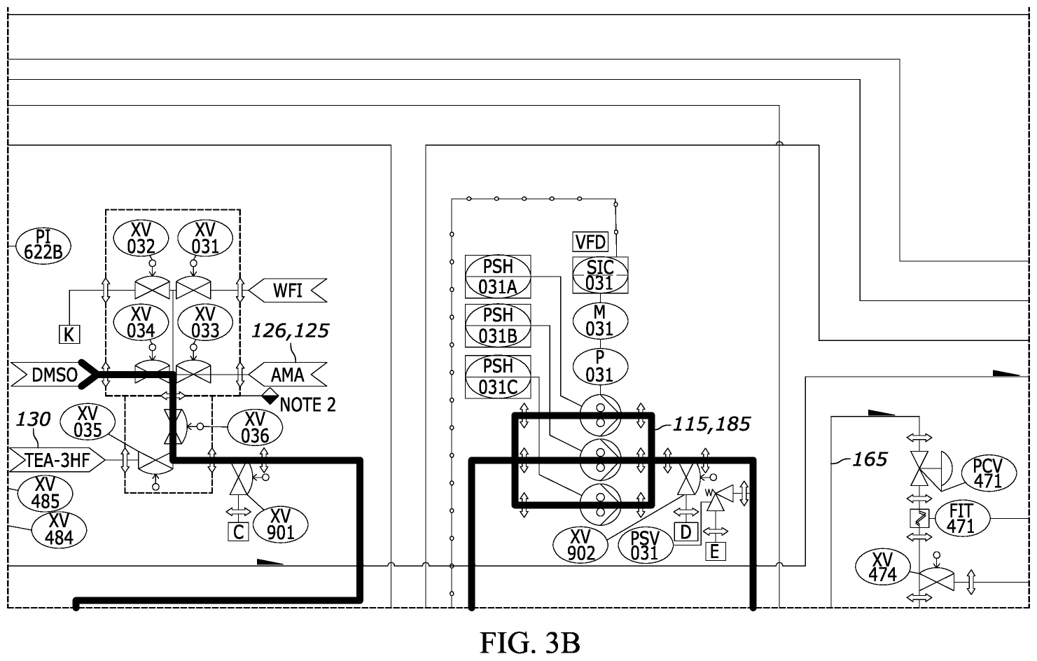

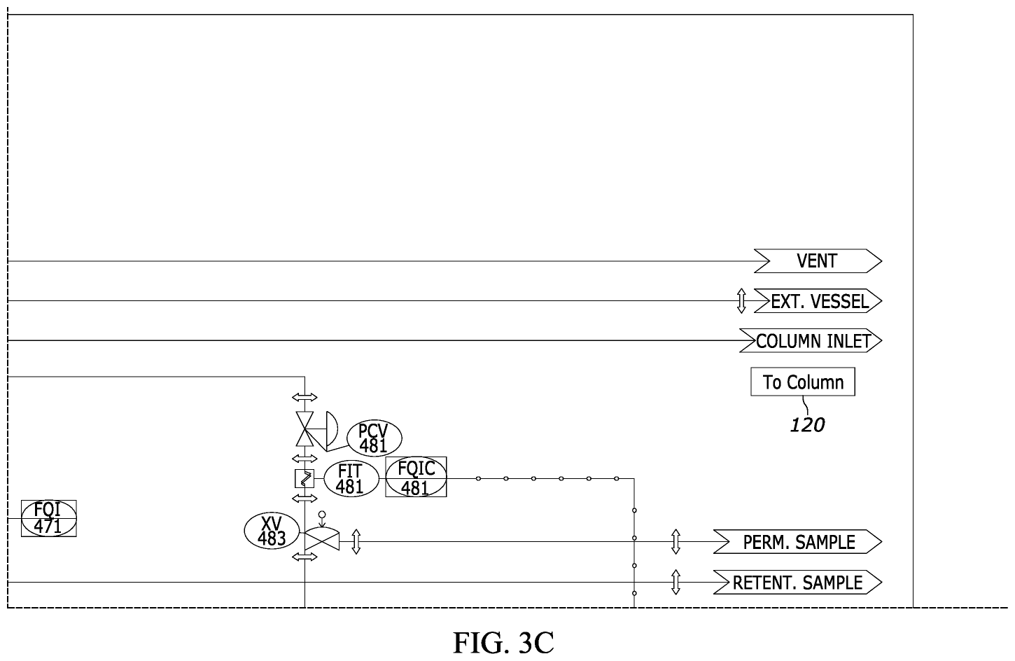

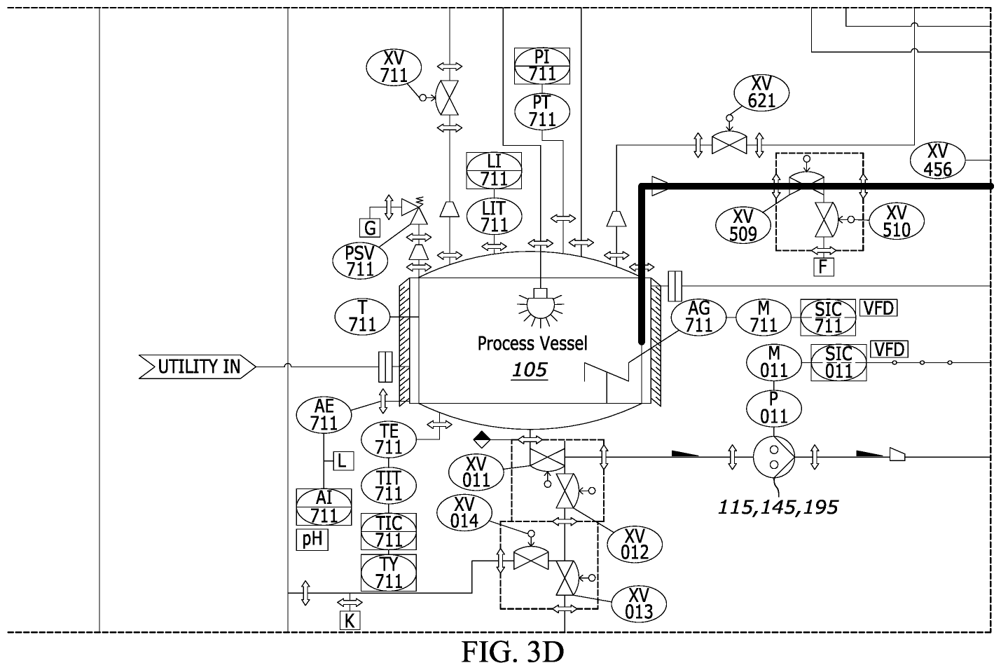

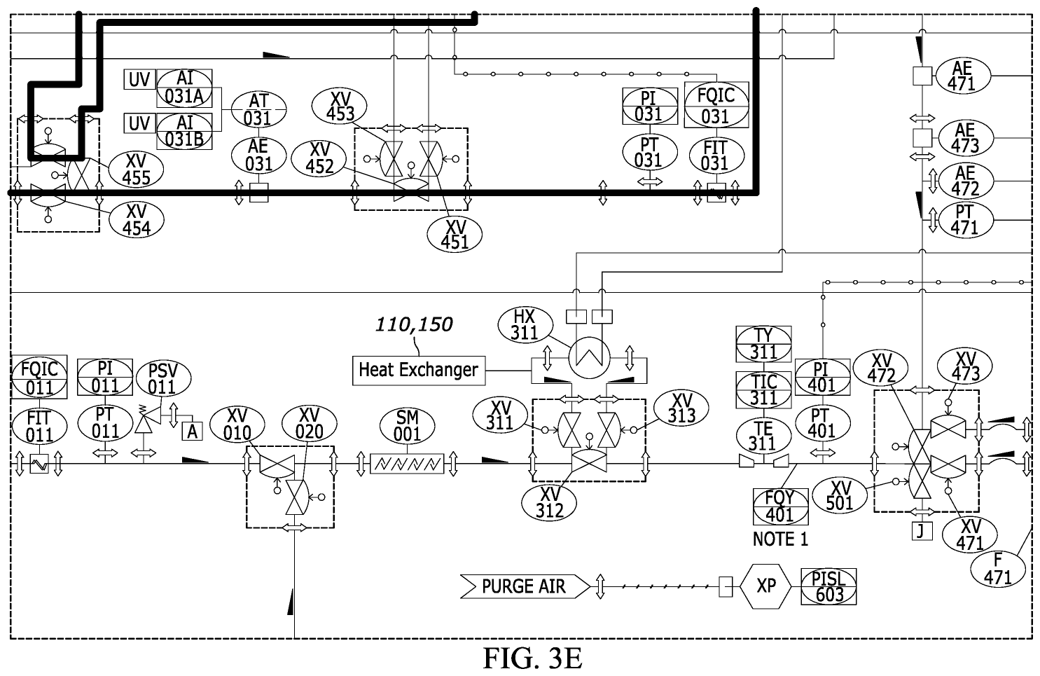

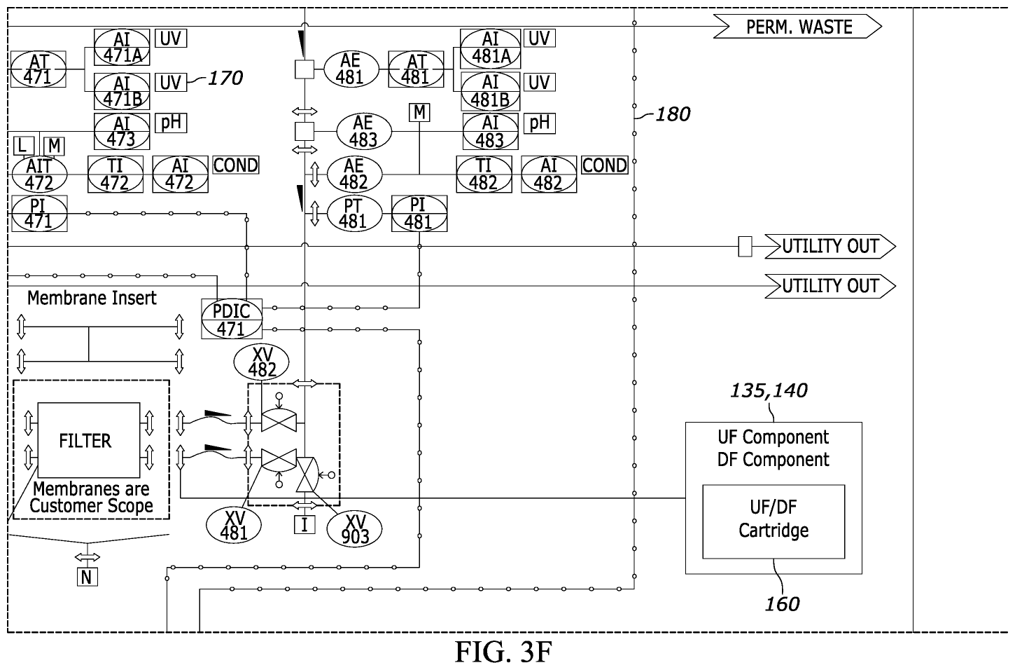

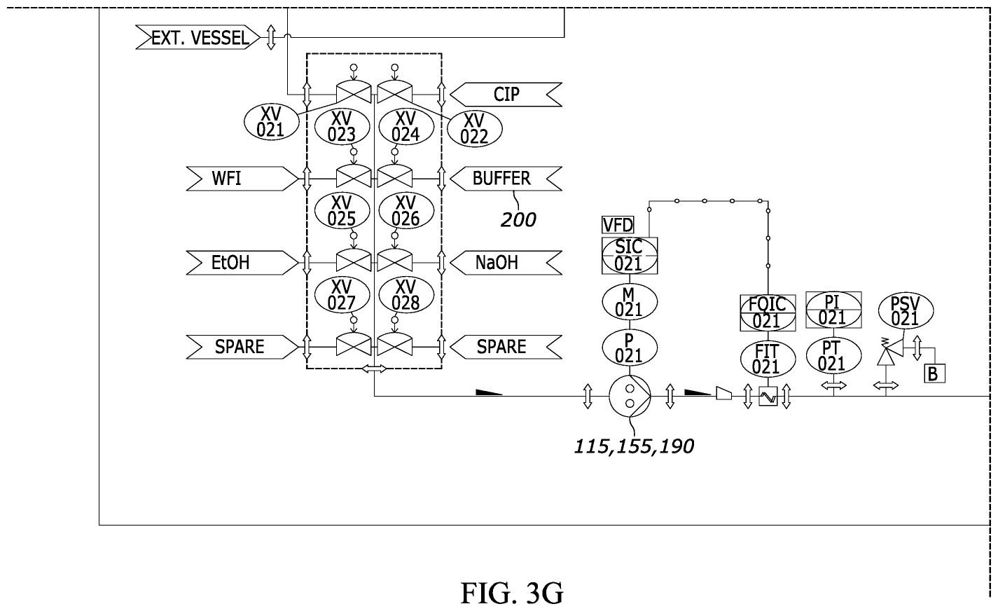

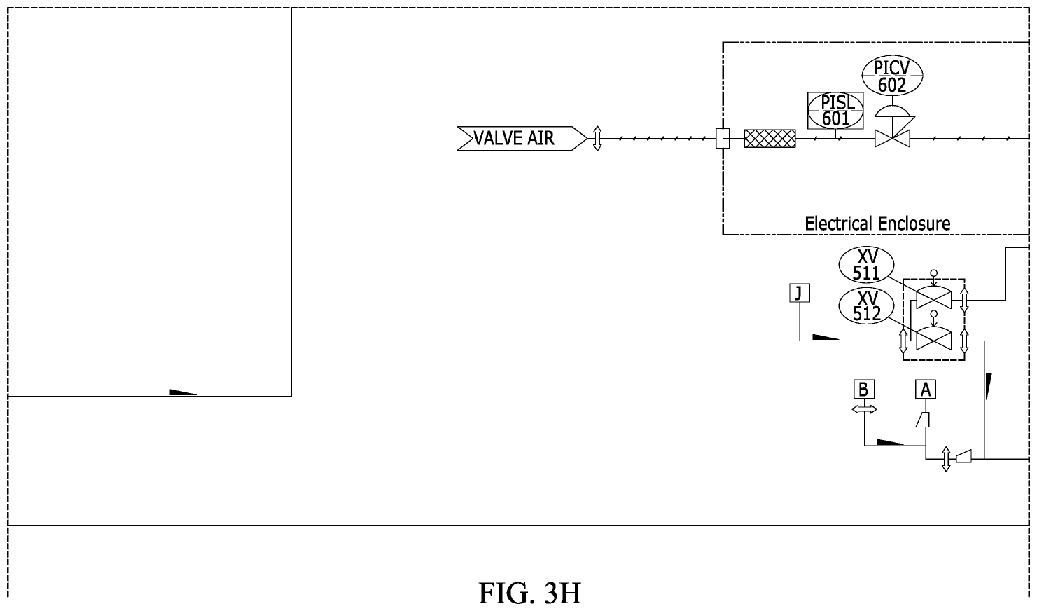

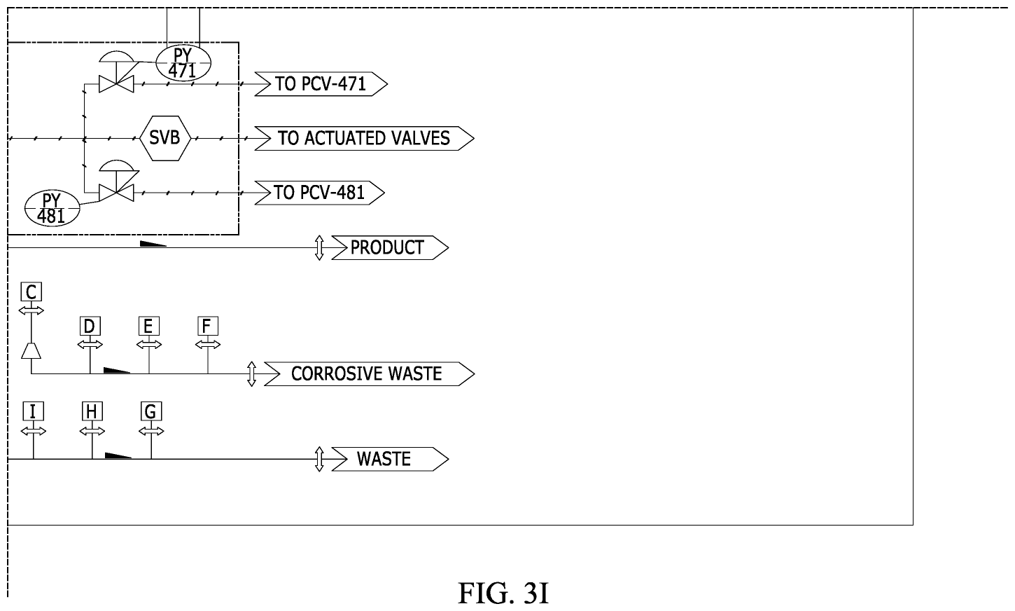

Turning now to FIGS. 3A-6I, which illustrate how the deprotection step is performed using the common system 100, the deprotection step is generally accomplished by pumping the deprotection component 130 from a supply through the deprotection pump 185 to the process vessel 105 and pumping the product solution from the process vessel 105 through the recirculation pump 145 and heat exchanger and back into the process vessel, thereby removing the chemical protecting groups previously used to prevent the chains from assembling incorrectly.

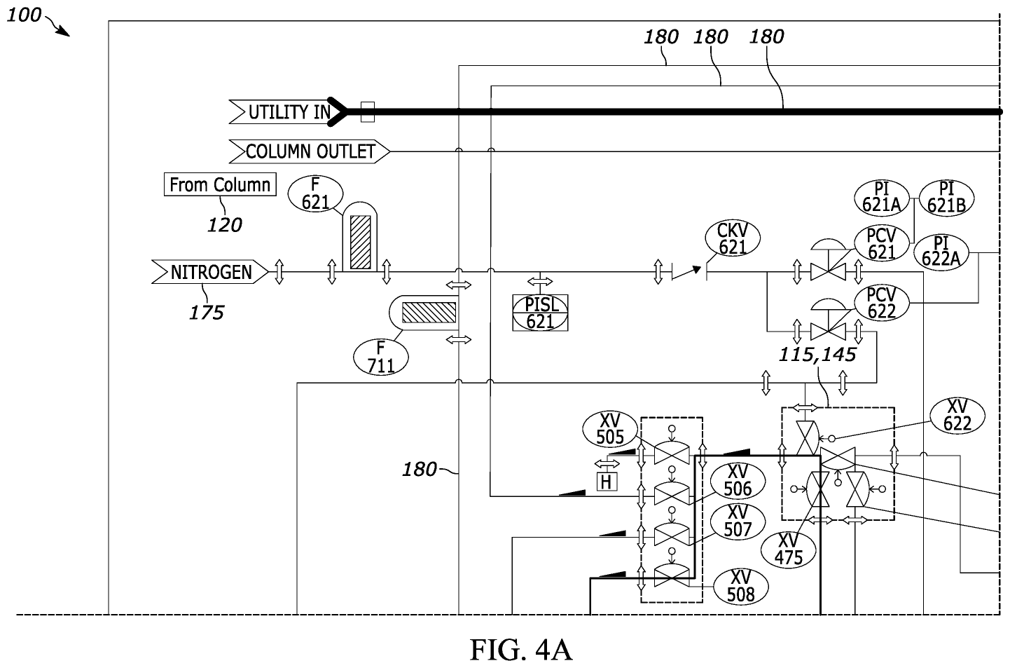

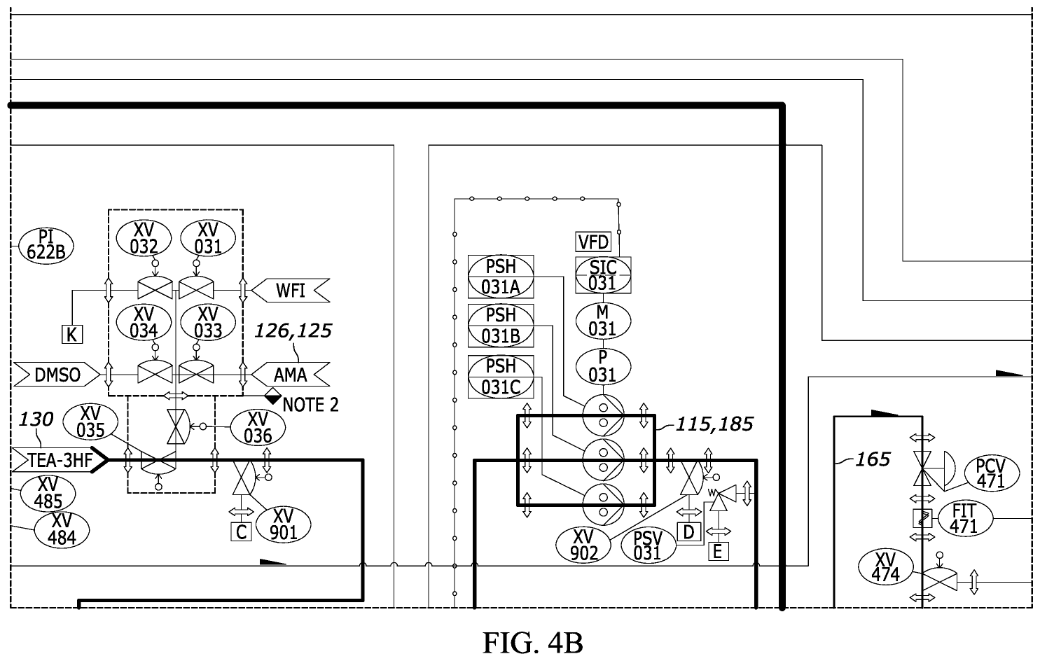

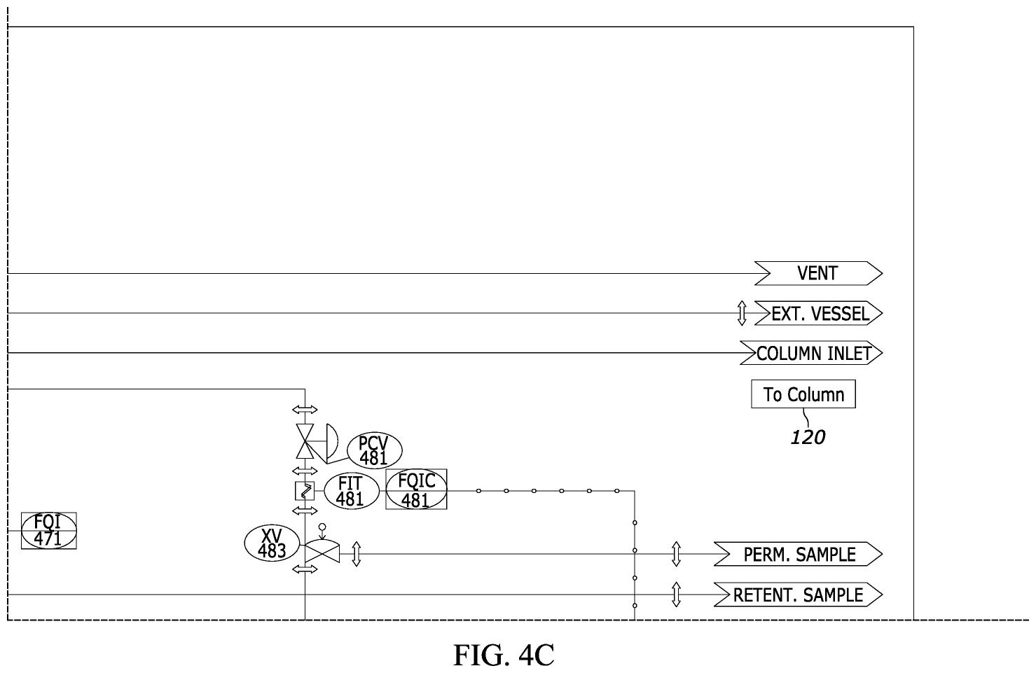

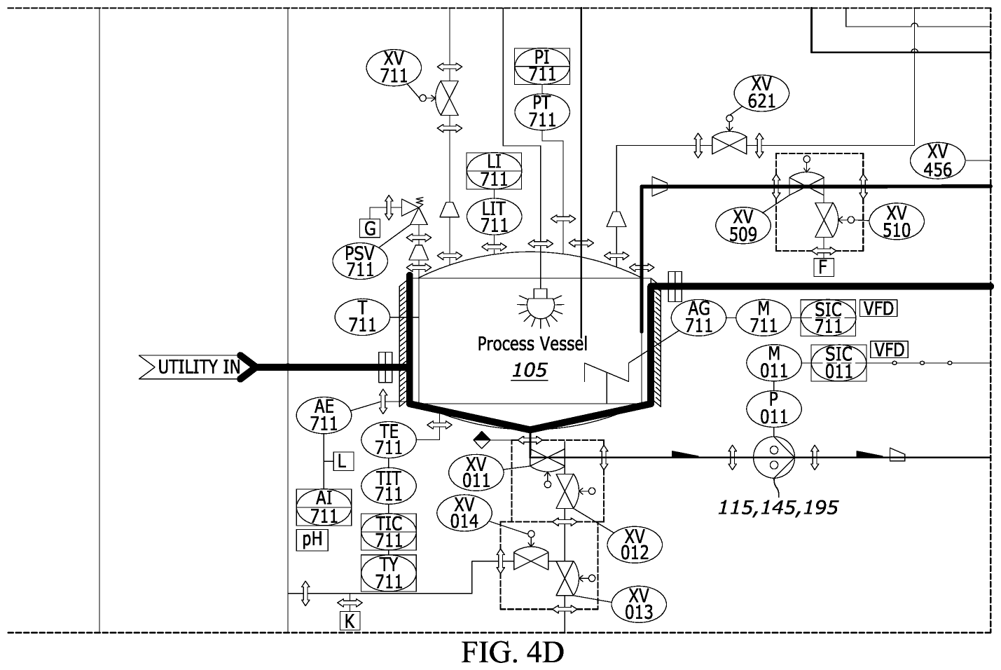

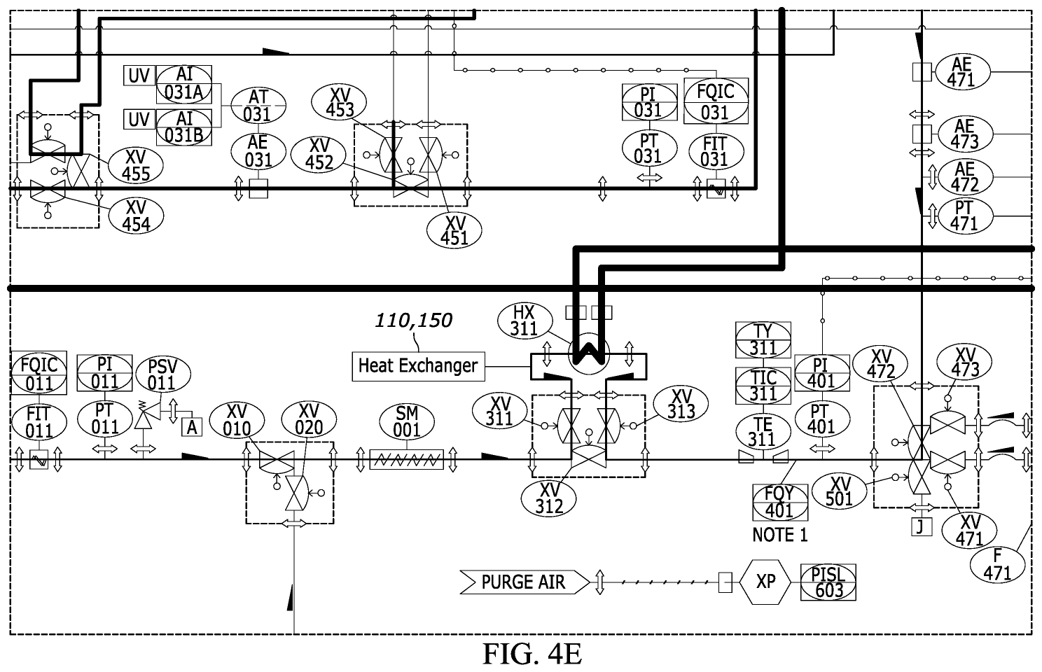

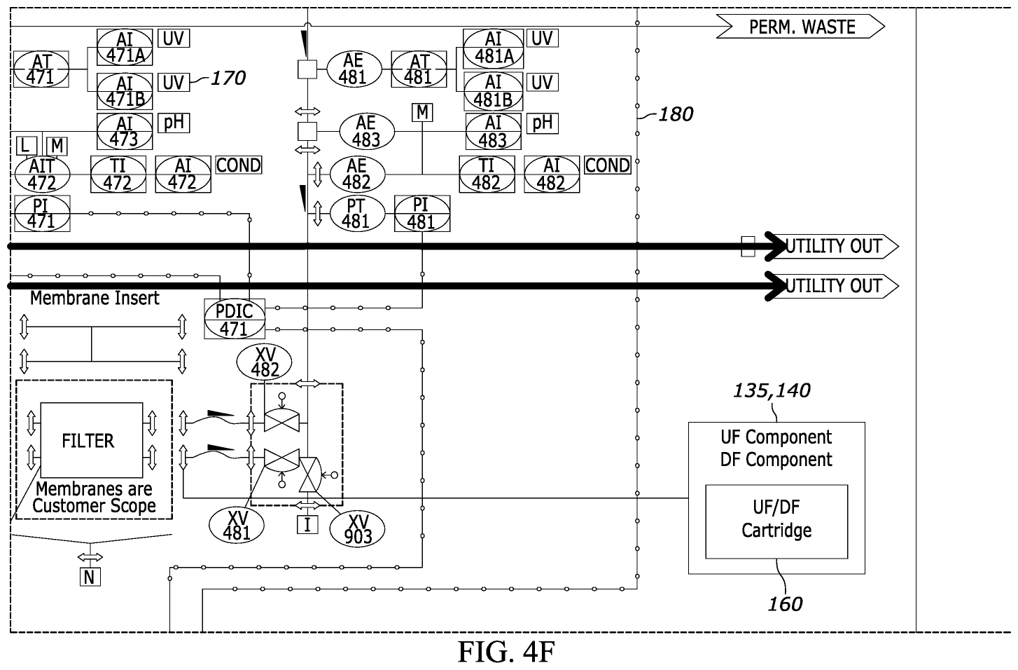

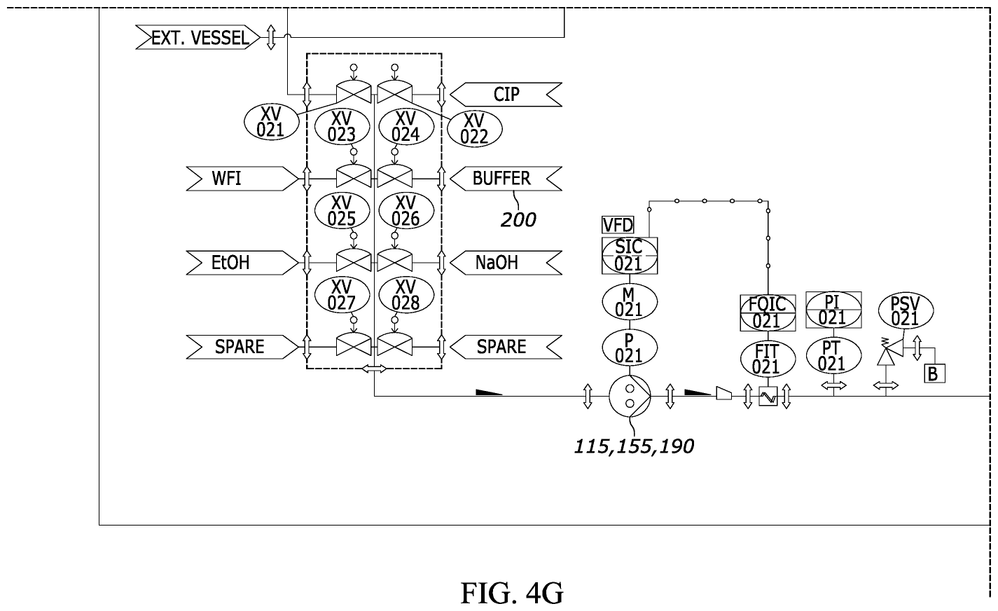

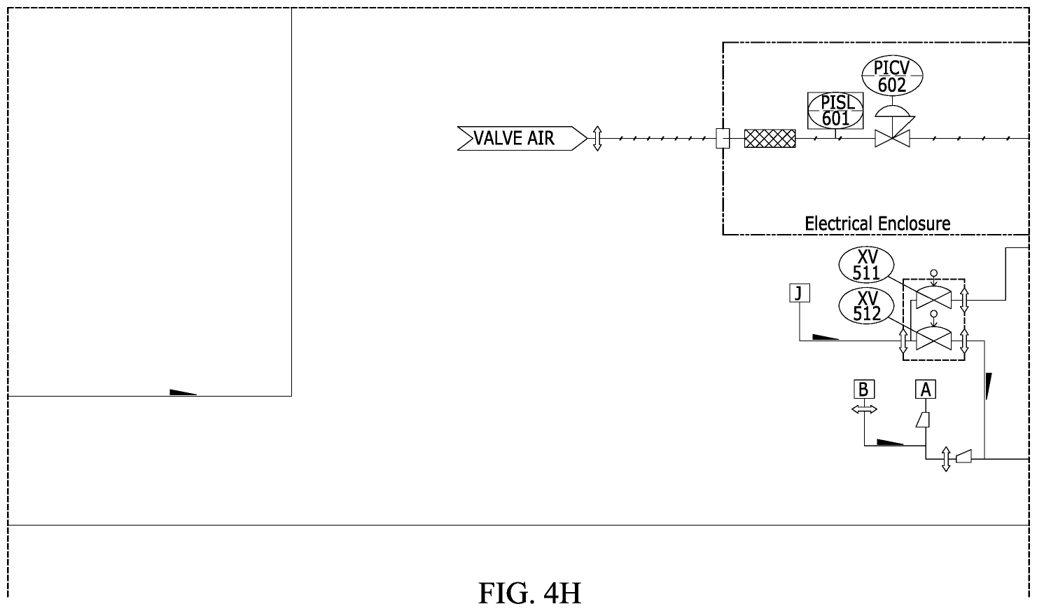

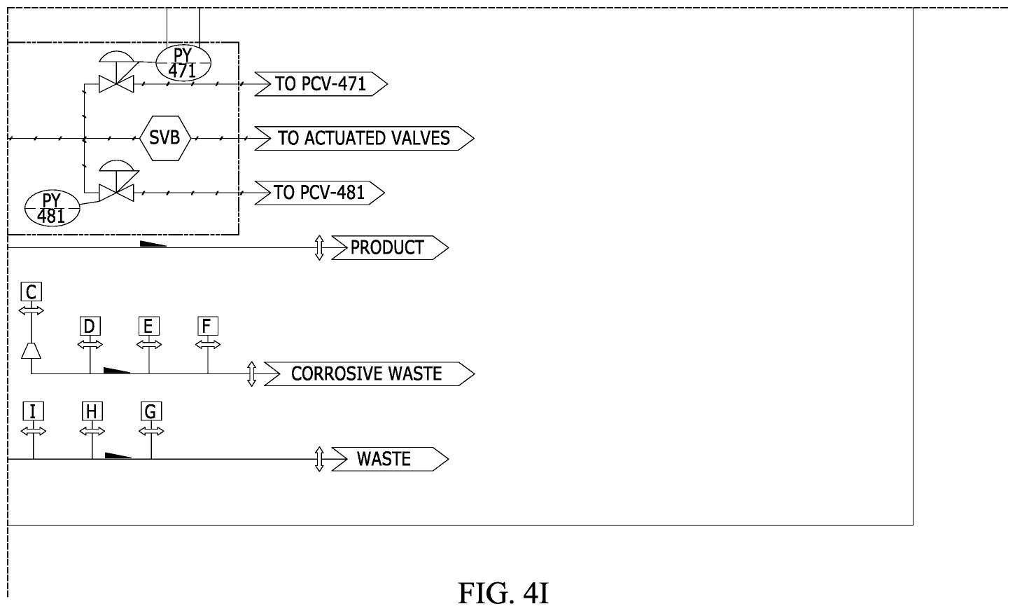

More particularly, after the oligo product is cleaved from the solid support, the oligo product may need to be further deprotected, depending on whether the application is for DNA or RNA. It will be appreciated by those skilled in the art that RNA comprises ribose, having a 2′ hydroxyl group that is protected during solid-phase synthesis. In RNA oligo synthesis, this protecting group is removed during processing into the final oligo product via a deprotection step. For DNA applications, the cleaved oligo product may skip the deprotection step and advance to further processing, in which case the contents of the vessel are cooled to ambient temperature and subjected to ultrafiltration (see next step below). For RNA applications, however, further deprotection is required. The deprotection step is performed with the deprotection component 130, which in this embodiment comprises a deprotection solution and the deprotection pump 185. In some embodiments, the deprotection solution is delivered to the process vessel 105 via the deprotection pump 185, as illustrated in FIGS. 3A-3I. In some embodiments, the deprotection component 130 further comprises a recirculation pump 145. In some embodiments, the deprotection pump 185 is configured to pump the cleavage solution from a supply thereof, through the oligo column 120, and into the process vessel 105, thereby flushing the oligo product from the oligo column into the process vessel. In some embodiments, the deprotection pump 185 is configured to pump the cleavage solution from a supply thereof, to recirculate the cleavage solution through the oligo column 120, and into the process vessel 105. In some embodiments, the deprotection pump 185 is further configured to pump a solvent and/or the deprotection solution from a supply thereof and into the process vessel 105, thereby mixing the solvent and/or the deprotection solution into the oligo product. In some embodiments, delivering the deprotection solution comprises dosing the deprotection solution into the process vessel 105. In some embodiments, the deprotection solution comprises a source of fluoride ion. In some embodiments, the deprotection step comprises adding a solvent (e.g., dimethyl sulfoxide (DMSO)) to the process vessel 105. In some embodiments, the deprotection solution comprises a deprotection reagent such as TEA-3HF. In some embodiments, the deprotection solution comprises TEA-3HF and a solvent, e.g., DMSO. In one embodiment, a solution of, for example, dimethyl sulfoxide (DMSO) is introduced to the vessel, and the contents of the vessel are cooled in preparation for the next addition. In this example, triethylamine trihydrofluoride (TEA-3HF) is then dosed intermittently into the vessel. This addition is highly exothermic and requires careful timing and concurrent cooling to keep the process from overheating. However, recirculation of the product solution and use of the inline heat exchanger 150 helps to retain tight temperature control over this critical process step. In some embodiments, the system comprises the recirculation pump 145, which is configured to pump a product solution comprising the oligo product from the process vessel 105 through the recirculation loop 165 and back into the process vessel, as illustrated in FIGS. 4A-4I.

In embodiments where a deprotection step is performed, addition of the deprotection reagent is performed under controlled temperature conditions. In some nonlimiting embodiments, the deprotection reagent comprises TEA-3HF. In some nonlimiting embodiments, TEA-3HF is added to the process vessel 105 under controlled temperature conditions to perform the deprotection step. Temperature control of the deprotection step can be achieved through various means known to those skilled in the art, for example, by pre-cooling the process vessel contents before deprotection reagent addition and/or dosing the deprotection reagent into the process vessel. In some embodiments, the process vessel contents are cooled to about −10° C. to −5° C. In some embodiments, the deprotection reagent is dosed into the process vessel continuously or intermittently to maintain the vessel contents below a specified temperature. In one nonlimiting embodiment, the process vessel contents are cooled to about −10° C. to −5° C. and TEA-3HF is dosed into the process vessel 105 continuously or intermittently at a rate that keeps the vessel contents below about 30° C. In some embodiments, the temperature of the process vessel 105 is maintained at about 10° C. or below. In some embodiments, maintaining the temperature of the process vessel 105 comprises recirculating the contents of the process vessel through an in-line temperature control element, e.g., an in-line heat exchanger. It is preferable that the deprotection reagent be accomplished as expediently as possible.

Once the deprotection solution (e.g., TEA-3HF) dosing is complete, the contents of the vessel are heated to a specified temperature for a specified duration, which breaks the molecule's 2′ bonds. In some embodiments, the process vessel contents are heated to about 25-50° C. In some embodiments, the process vessel contents are heated for about 3-5 hours. In one nonlimiting embodiment, after TEA-3HF addition to the process vessel 105 is complete, the process vessel contents are heated to about 25-50° C. for about 3-5 hours. In one nonlimiting embodiment, after TEA-3HF addition to the process vessel 105 is complete, the process vessel contents are heated to about 50° C. for about 3-5 hours.

The final step in the deprotection process involves quenching the solution after the deprotection solution (e.g., acid) addition. This can be accomplished by combining the contents of the process vessel 105 with a quenching buffer. In one example, combining the contents of the process vessel 105 and the quenching buffer comprises adding a quenching buffer to the vessel, mixing the quenching buffer with the contents of the vessel inline, adding the contents of the vessel to a different vessel containing the quenching buffer, or a combination of these strategies. Preferably, however, this step is accomplished by introducing the quench buffer via inline mixing concurrently with the start of the ultrafiltration step, thereby avoiding the need for a separate or larger vessel. In some embodiments, the system comprises a quenching feed pump 190 configured to pump the quenching buffer from a supply thereof 200 and into the process vessel 105, thereby combining the oligo product with the quenching buffer to form a combined product solutionI. Suitable quenching buffers will be known to those skilled in the art. In some embodiments, the quenching buffer is a TRIS buffer.

### Ultrafiltration

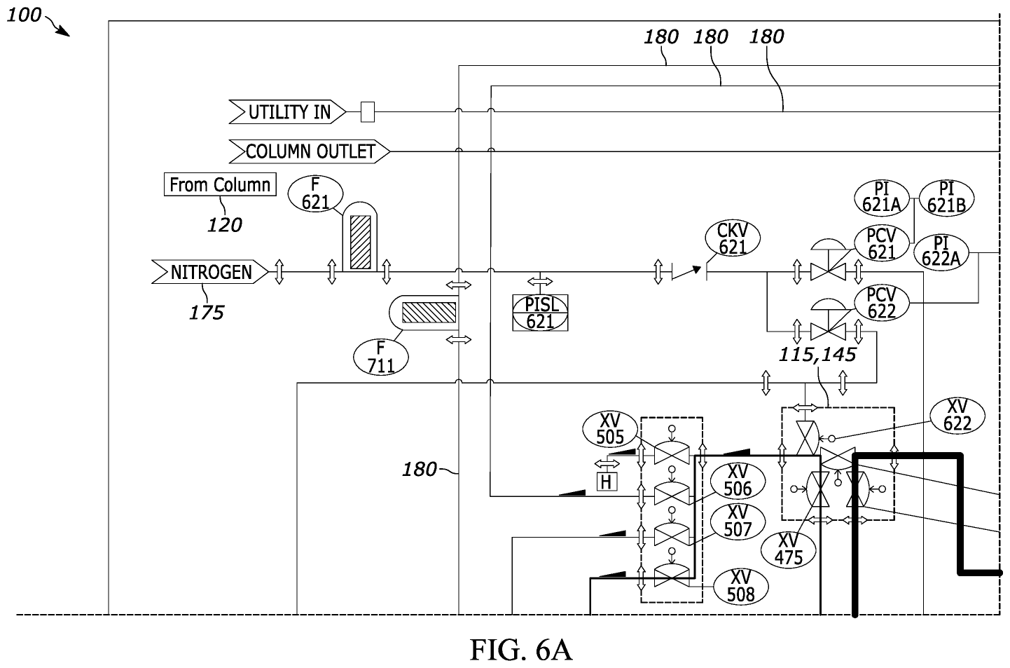

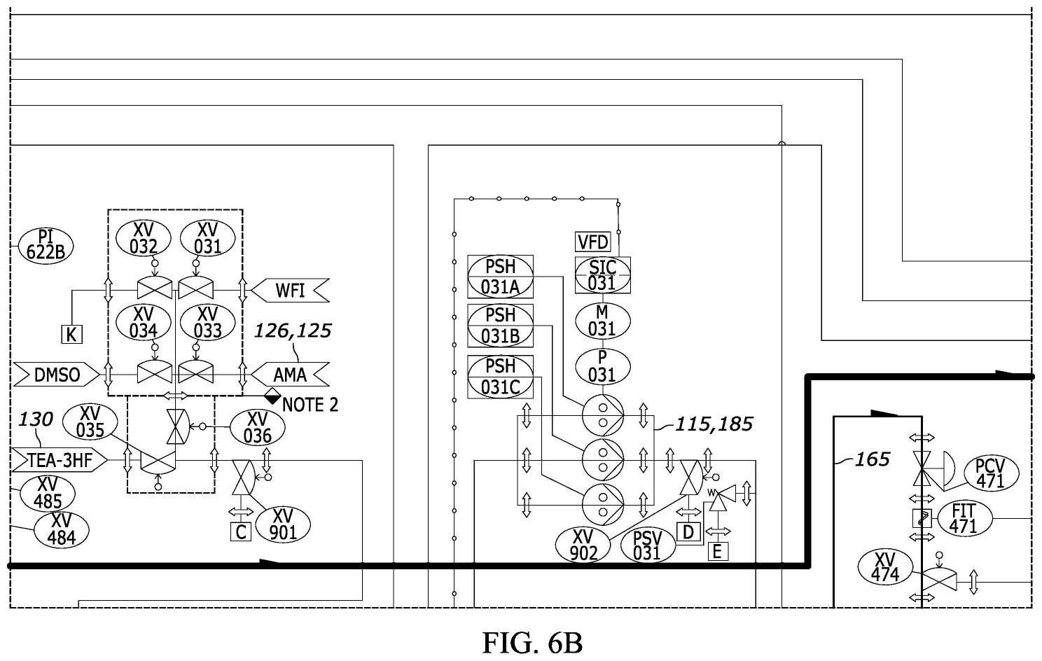

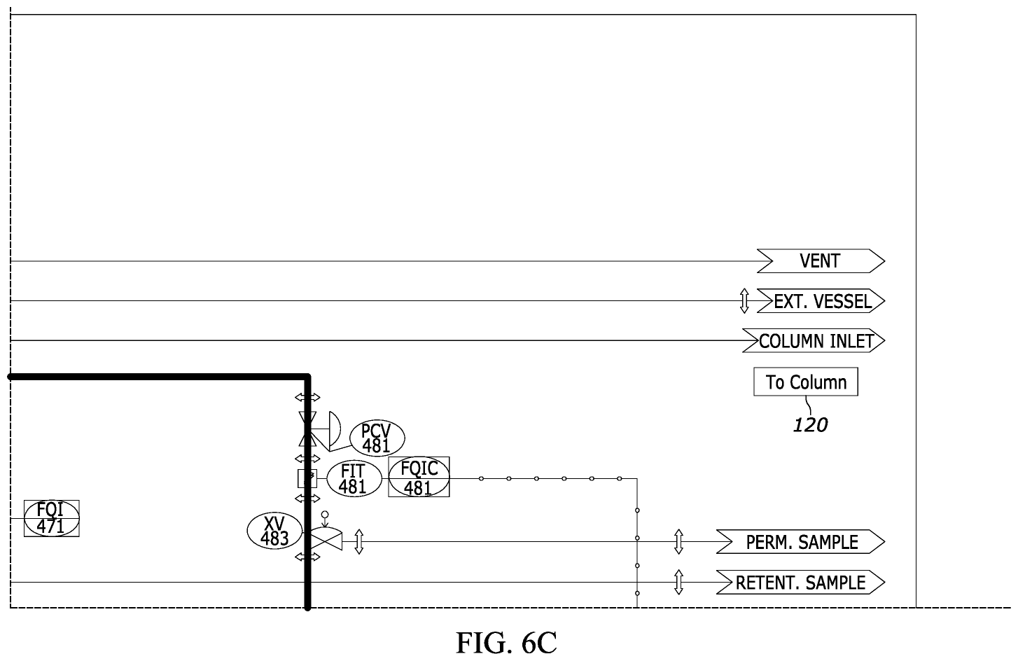

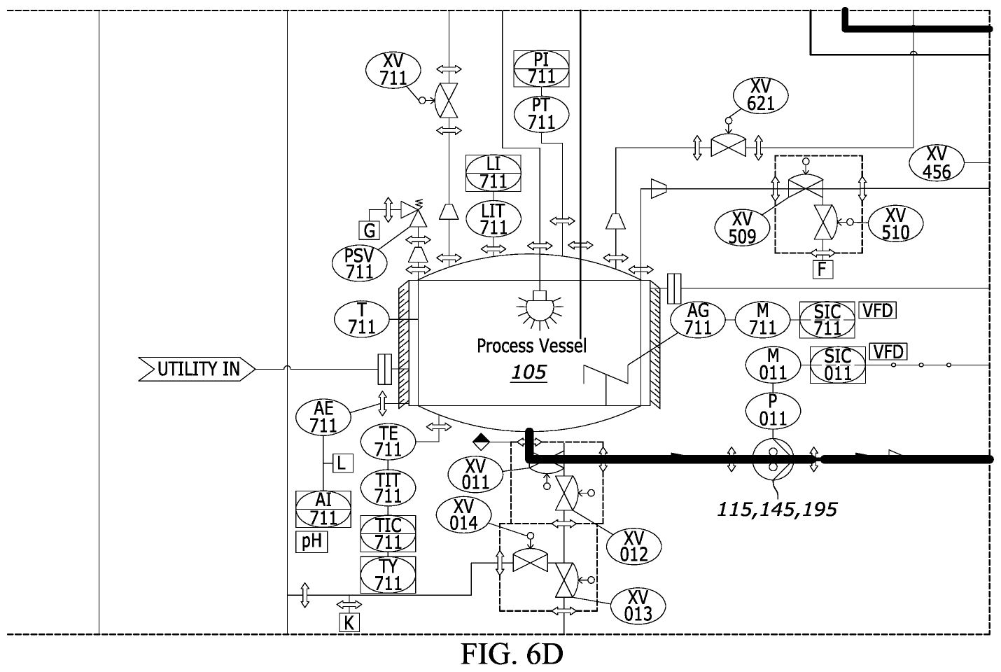

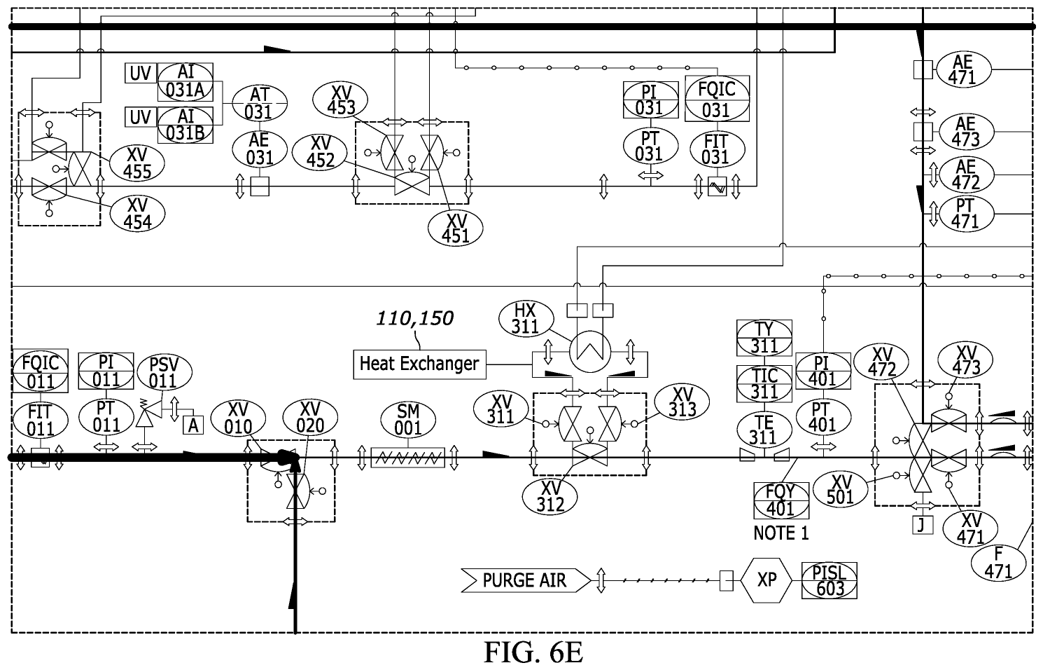

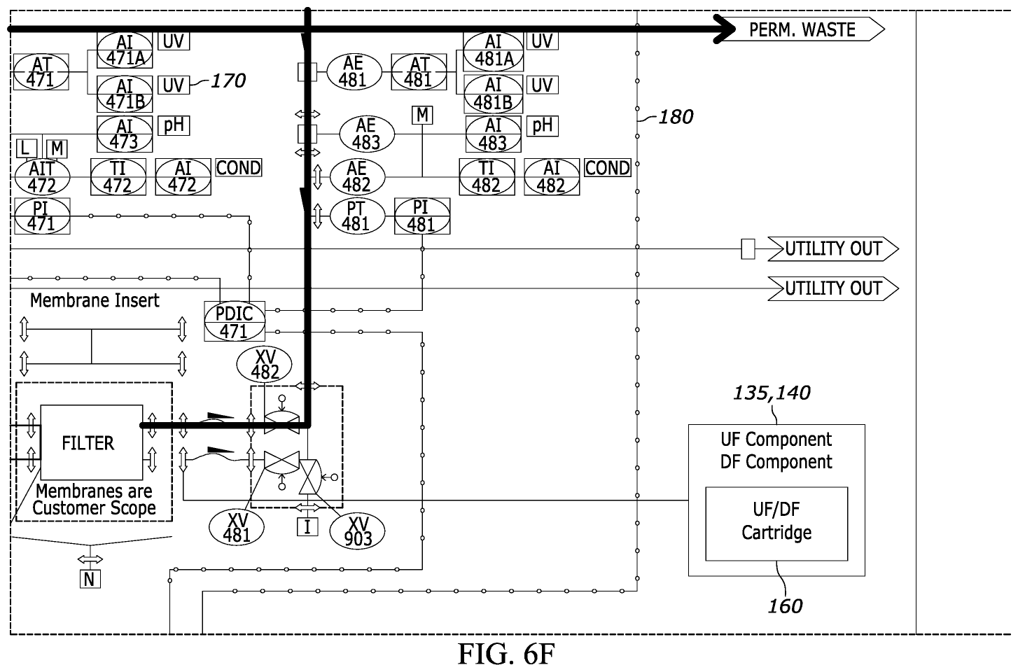

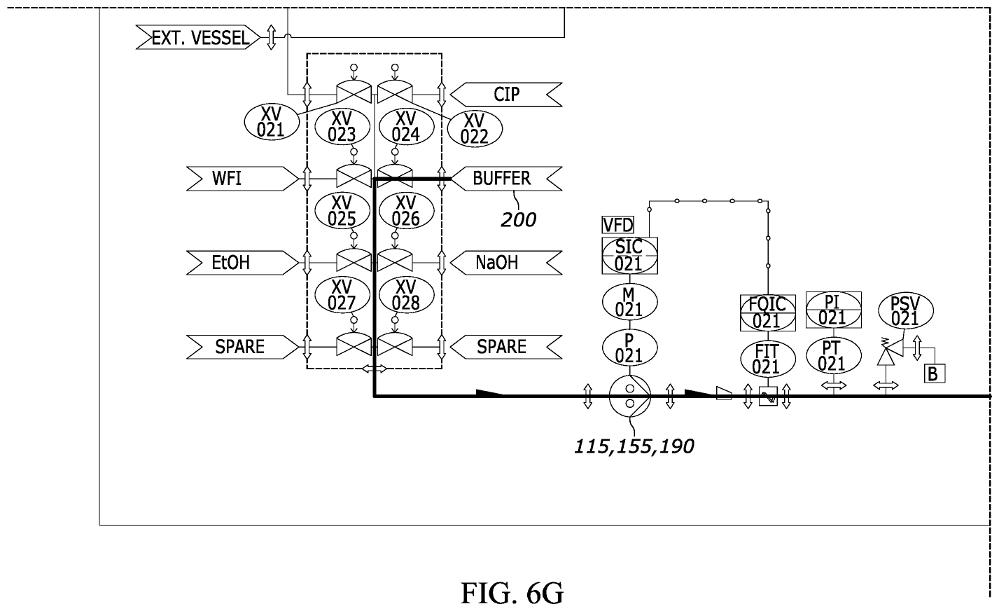

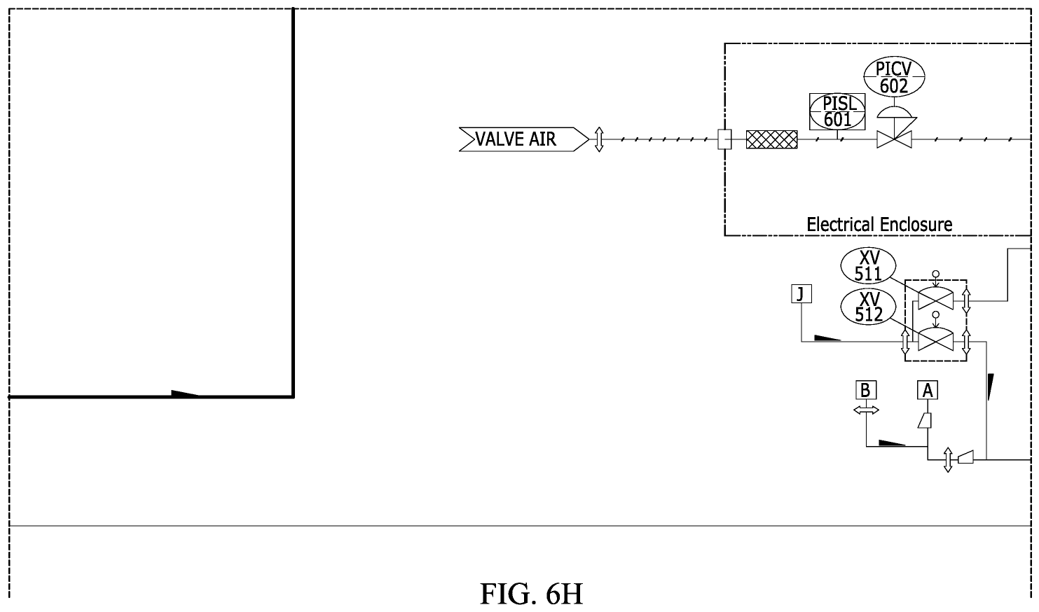

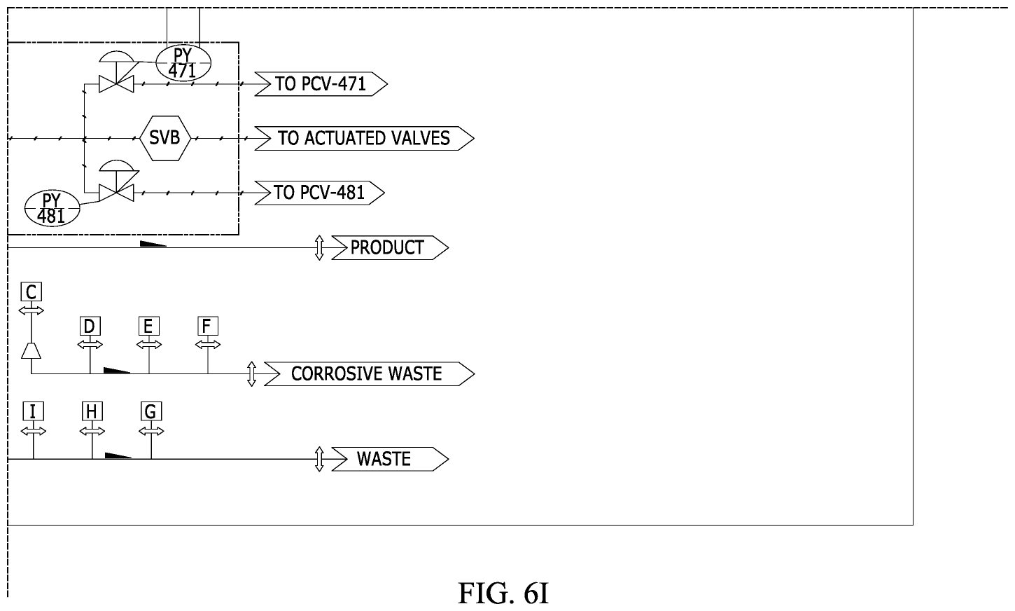

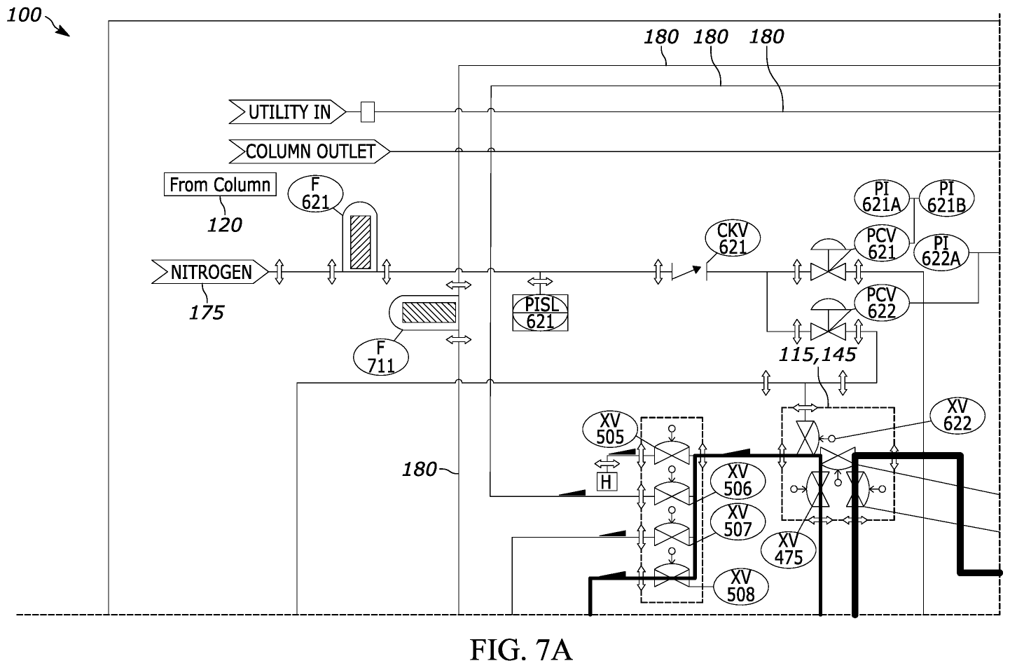

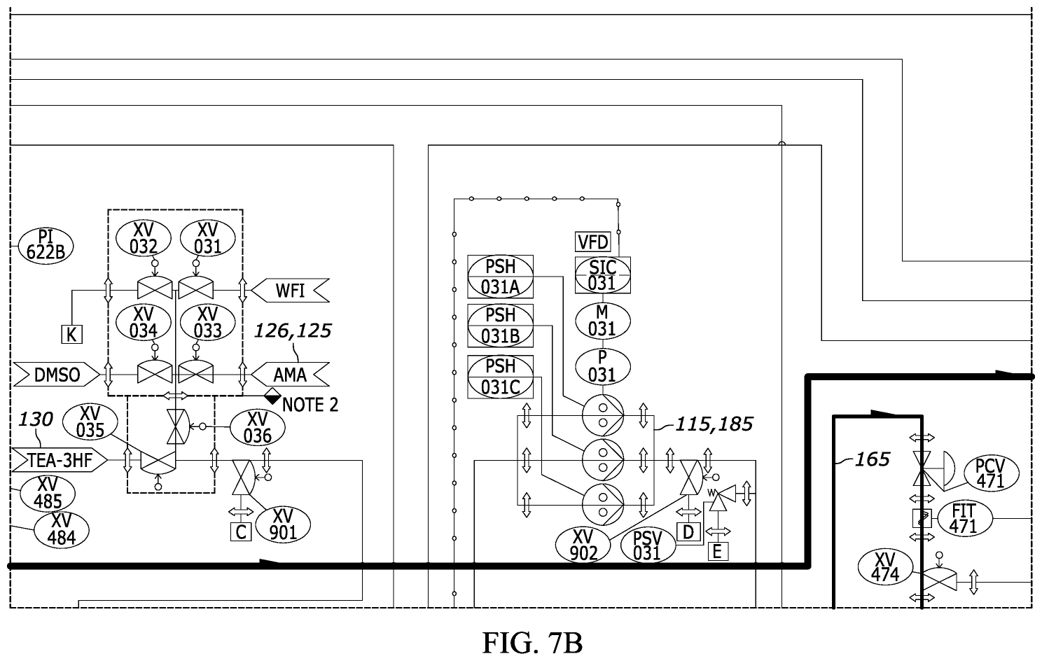

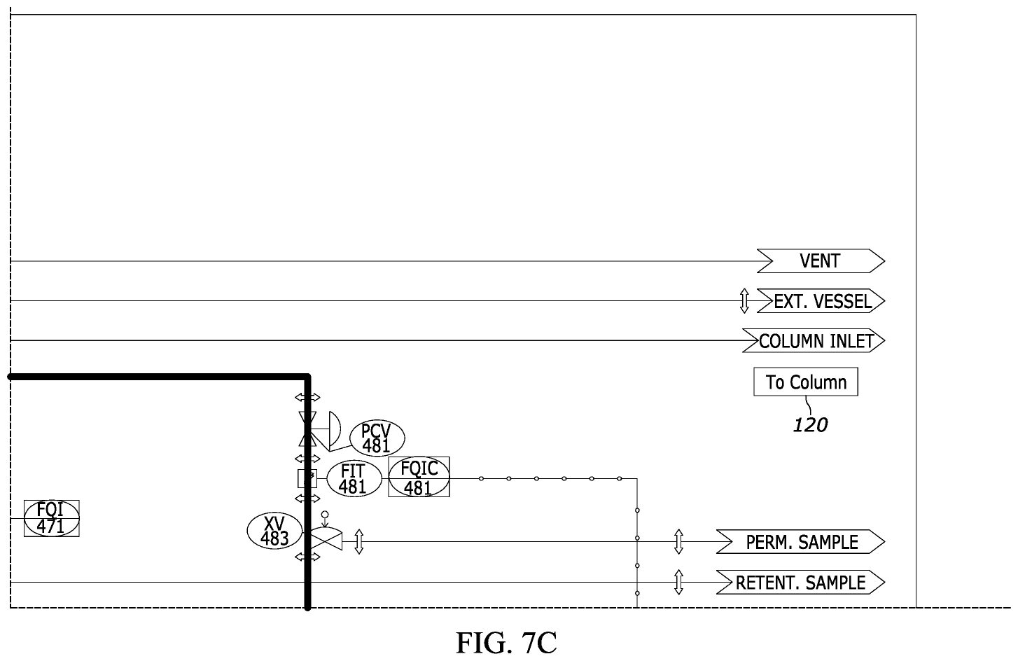

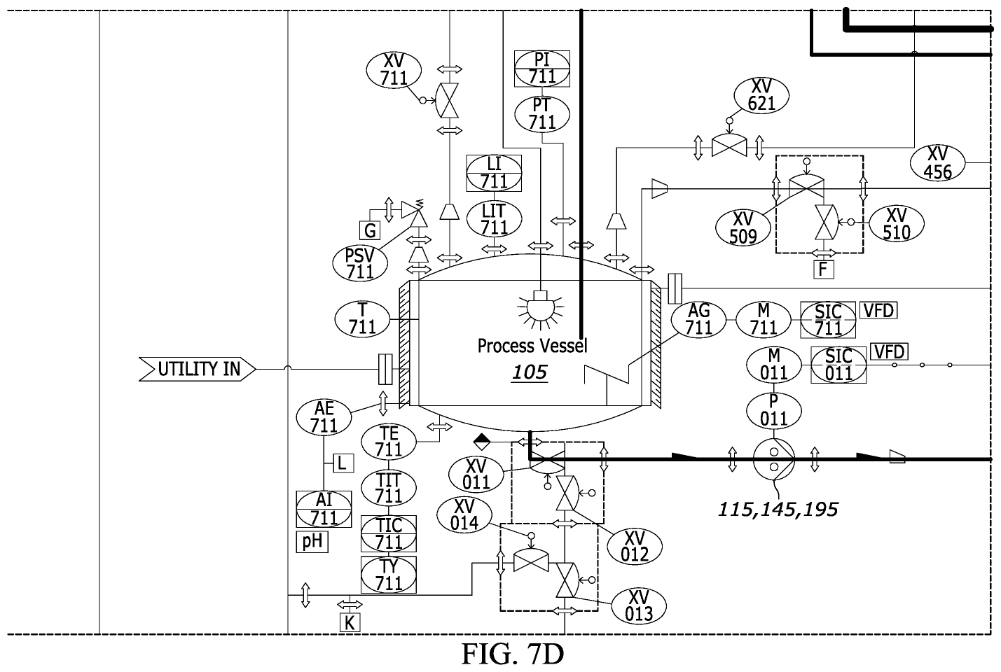

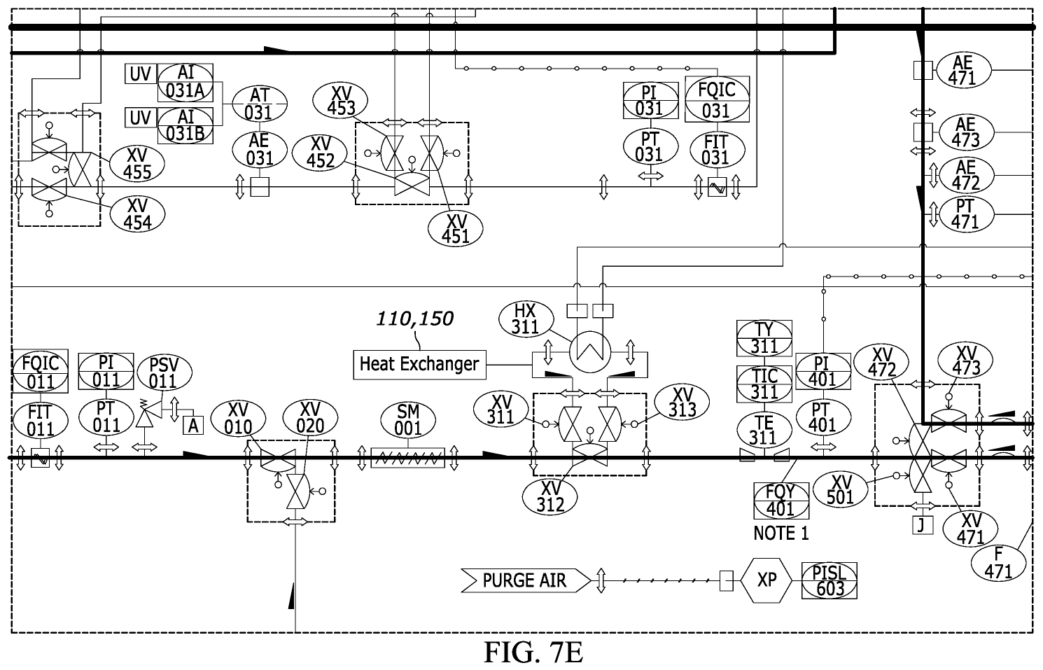

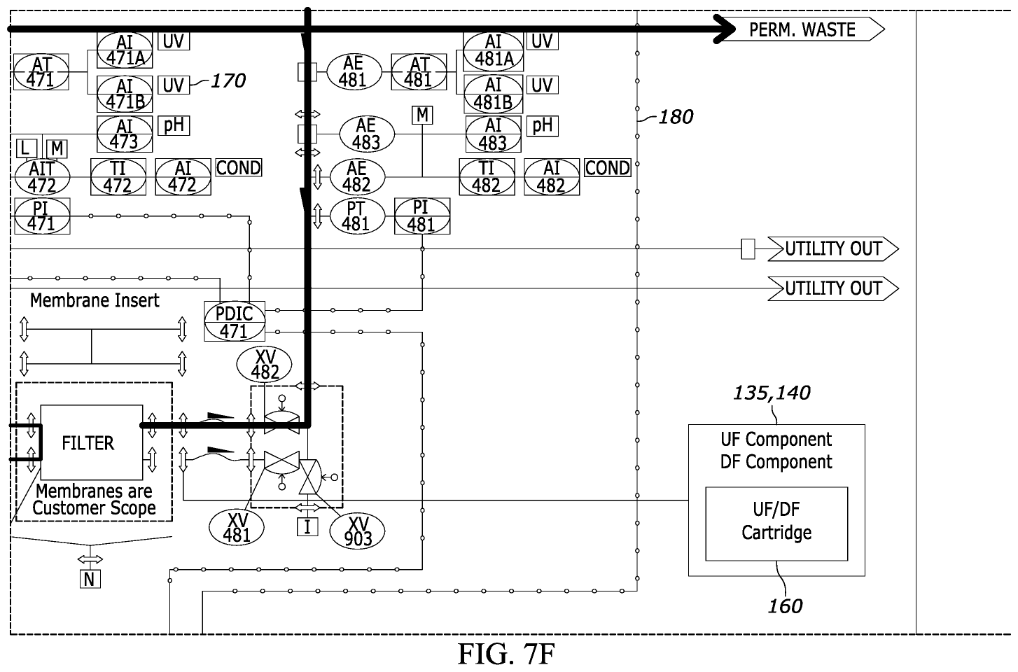

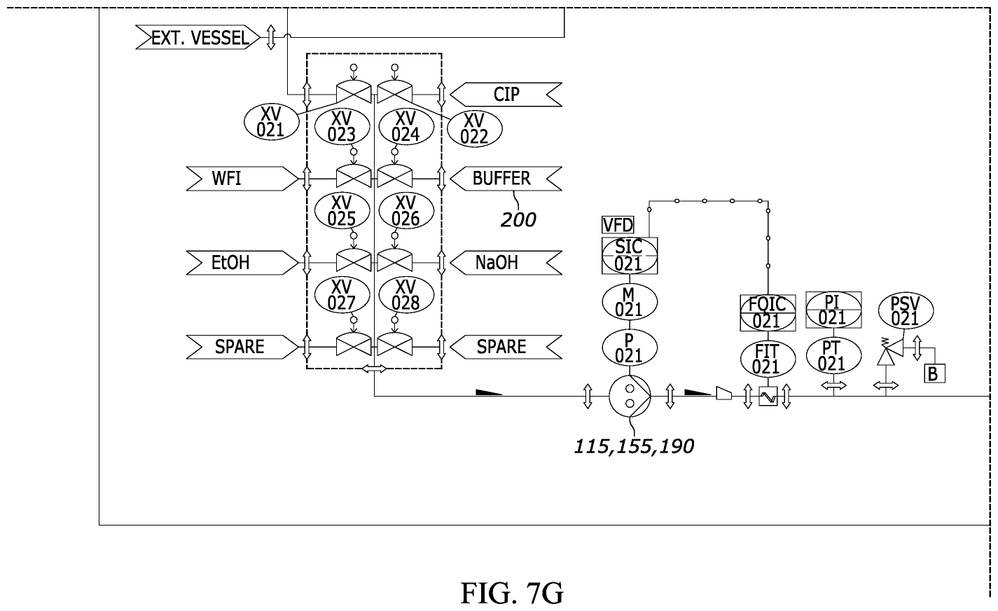

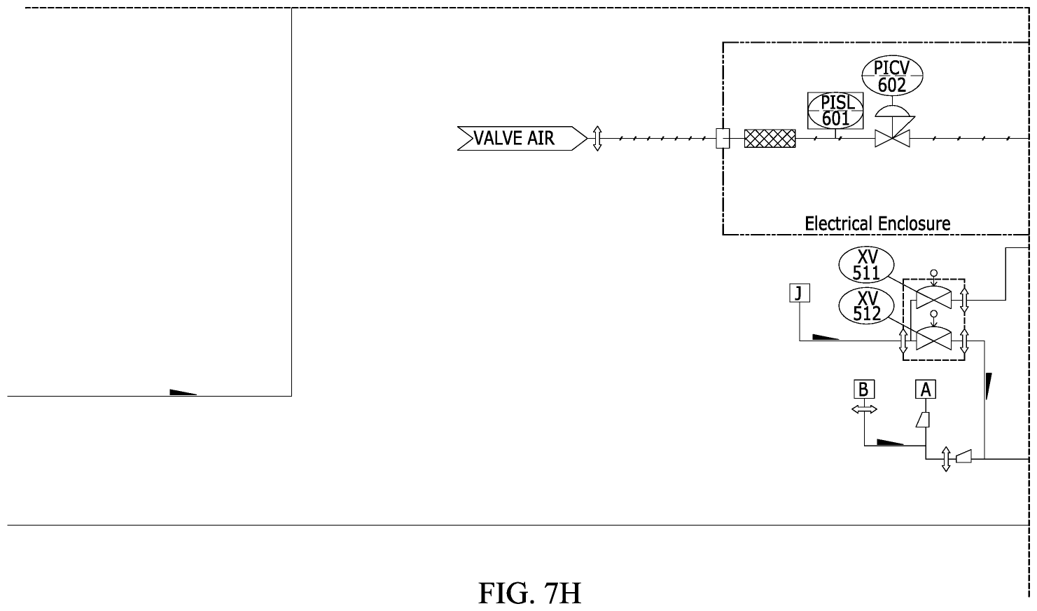

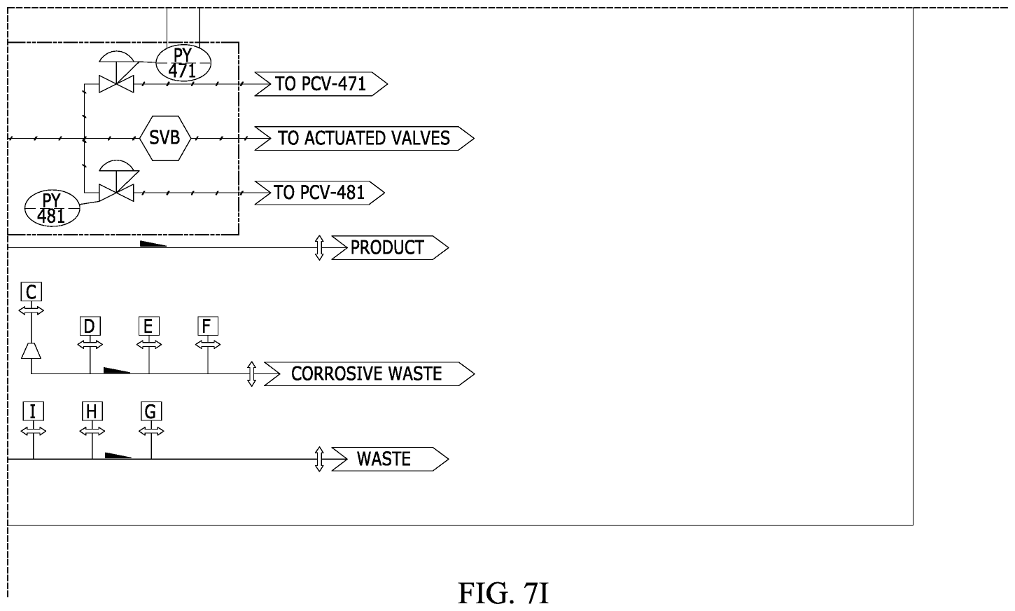

Turning now to FIGS. 6A-6I and 7A-7I, which illustrate how the ultrafiltration step is performed using the common system 100, the ultrafiltration operation is generally accomplished by pumping the product solution from the process vessel 105 through the recirculation pump 145 and the ultrafiltration component 135 and back into the process vessel 105 as the permeate is directed to waste, thereby concentrating the product solution. The ultrafiltration step is performed using the ultrafiltration component 135, which in this embodiment comprises an ultrafiltration membrane. Those skilled in the art will appreciate that various ultrafiltration strategies are possible, including passing the contents of the process vessel 105 across or through the ultrafiltration membrane depending on the nature of the membrane. With respect to the ultrafiltration membrane, the terms “across” and “through” are used interchangeably herein, and the skilled artisan will understand which term is applicable to a particular membrane.

More particularly, after the oligo product has been deprotected, the oligo product is concentrated using ultrafiltration. As briefly mentioned above, since the same system is capable of performing cleavage, deprotection, ultrafiltration, and diafiltration operations, the product does not need to be transferred to another location or machine. In some embodiments, the ultrafiltration component 135 and diafiltration component 140 together comprise the common ultrafiltration/diafiltration cartridge (“UF/DF cartridge”) 160. In some embodiments, the UF/DF cartridge 160 comprises a membrane capable of performing ultrafiltration and diafiltration. In one embodiment, employing tangential flow filtration (“TFF”), the oligo product is directed to the UF/DF cartridge 160 where it is exposed to a UF/DF membrane. The product remains on the retentate side of the membrane while impurities and salts pass through to the permeate side. This process is recirculated from the vessel through the recirculation loop 165 and the UF/DF cartridge 160. This reduces the total volume of the vessel contents as the product is concentrated. In some embodiments, the ultrafiltration component 135 and diafiltration component 140 comprise an inline mixer. In some embodiments, the common system 100 comprises a quenching recirculation pump 195 configured to pump the combined product solution and the quenching buffer from the process vessel 105 through the recirculation loop 165 and to the UF/DF cartridge 160, such that the combined product solution and the quenching buffer are exposed to the UF/DF membrane, thereby producing a first permeate that is directed to waste and a first retentate comprising the oligo product that flows back into the process vessel.

### Diafiltration

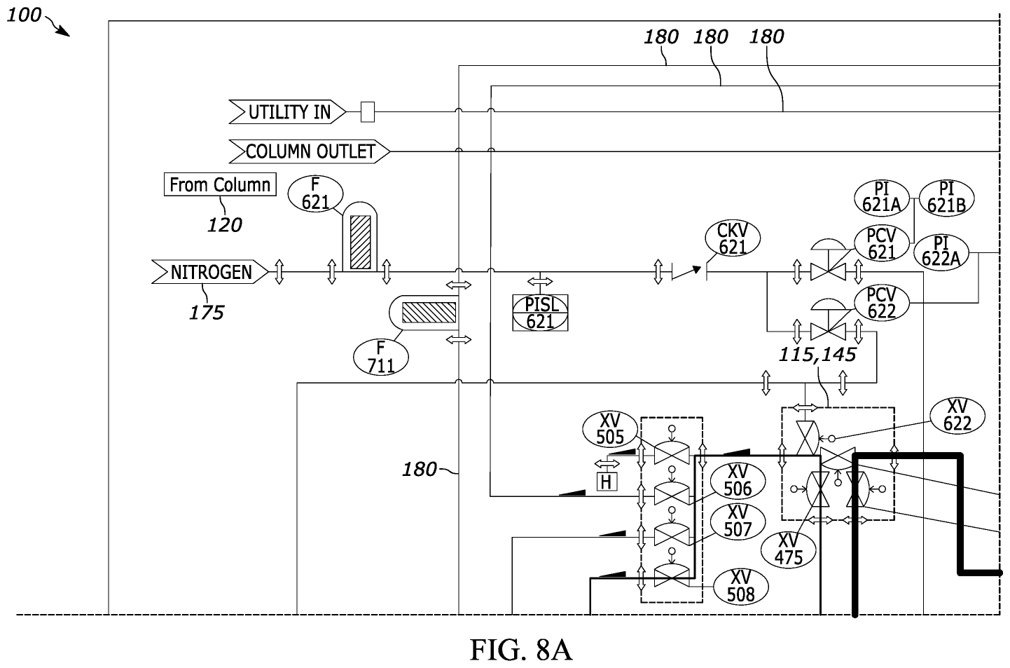

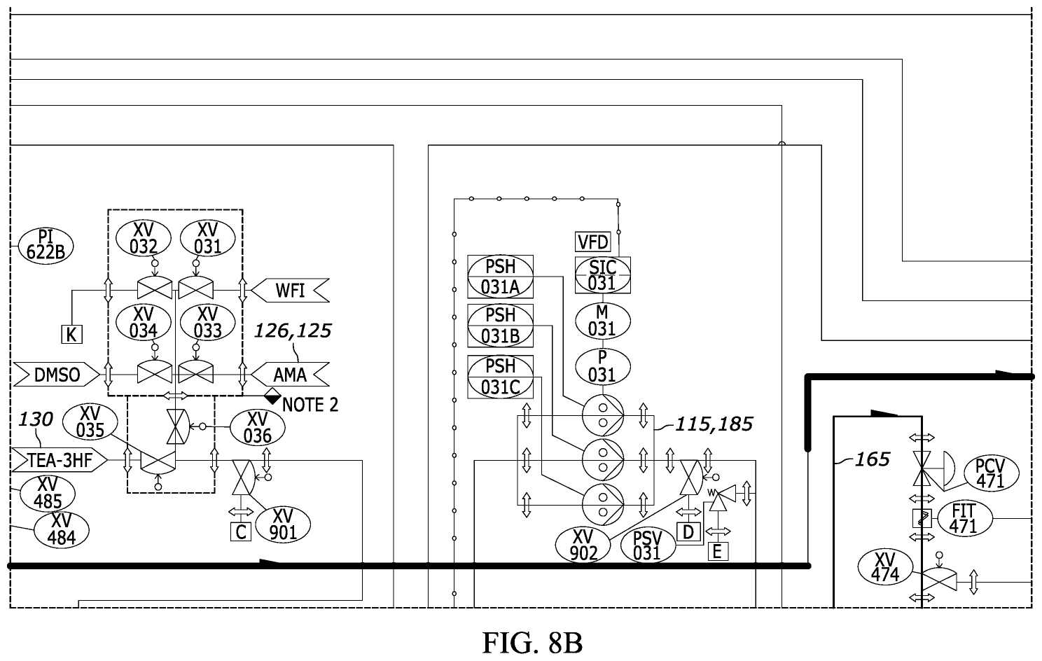

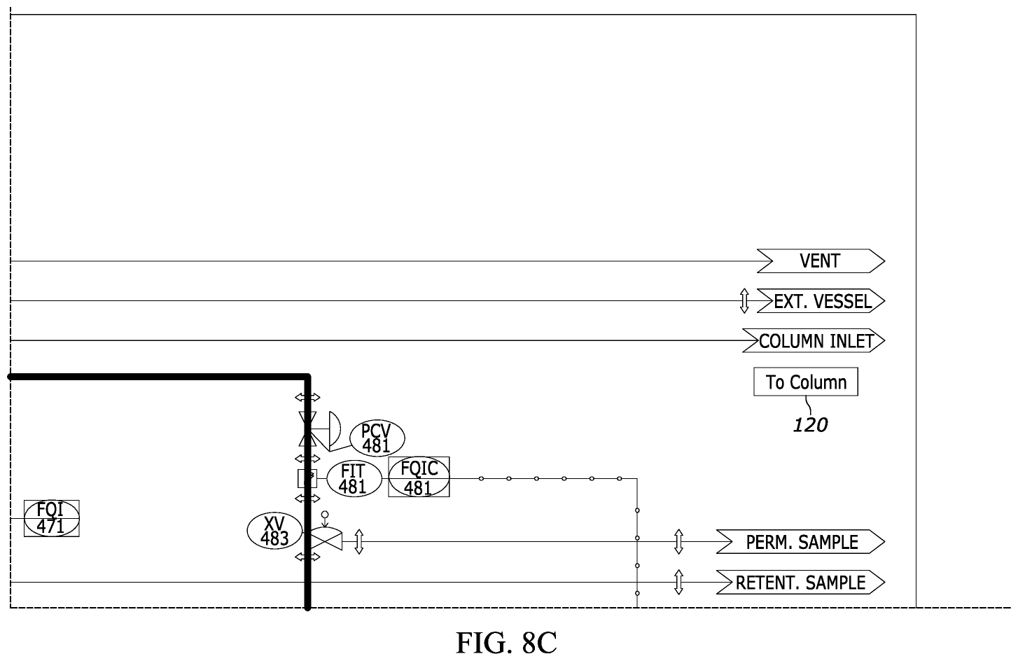

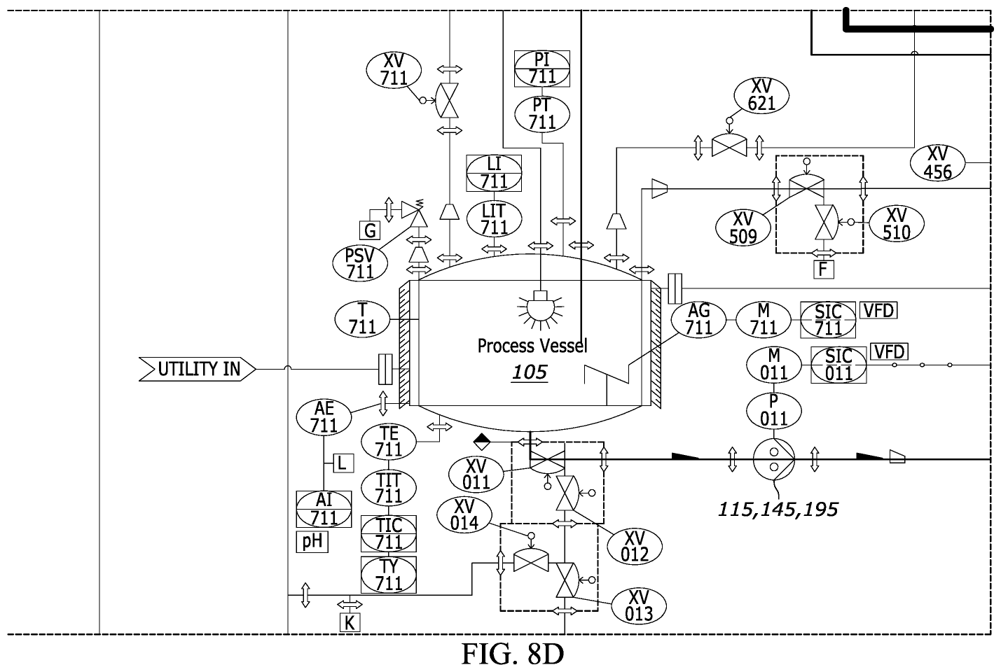

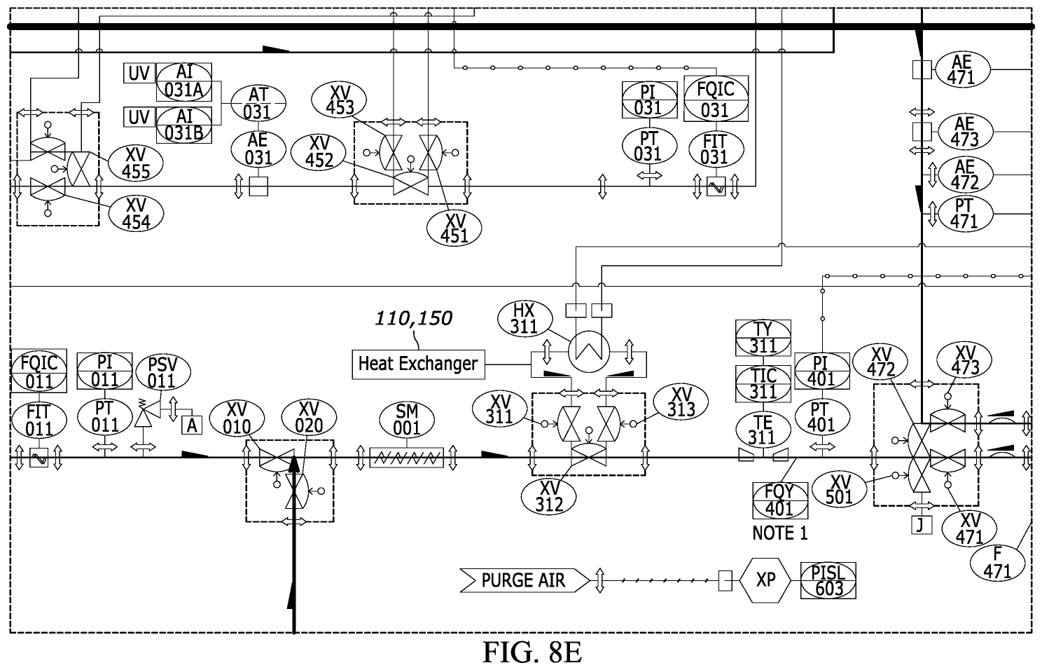

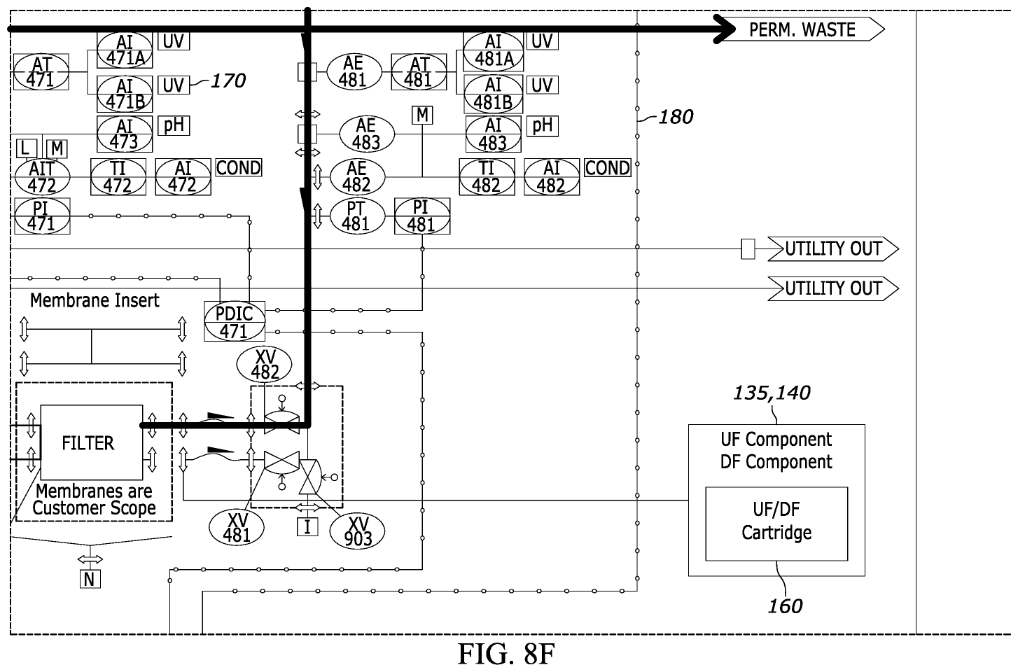

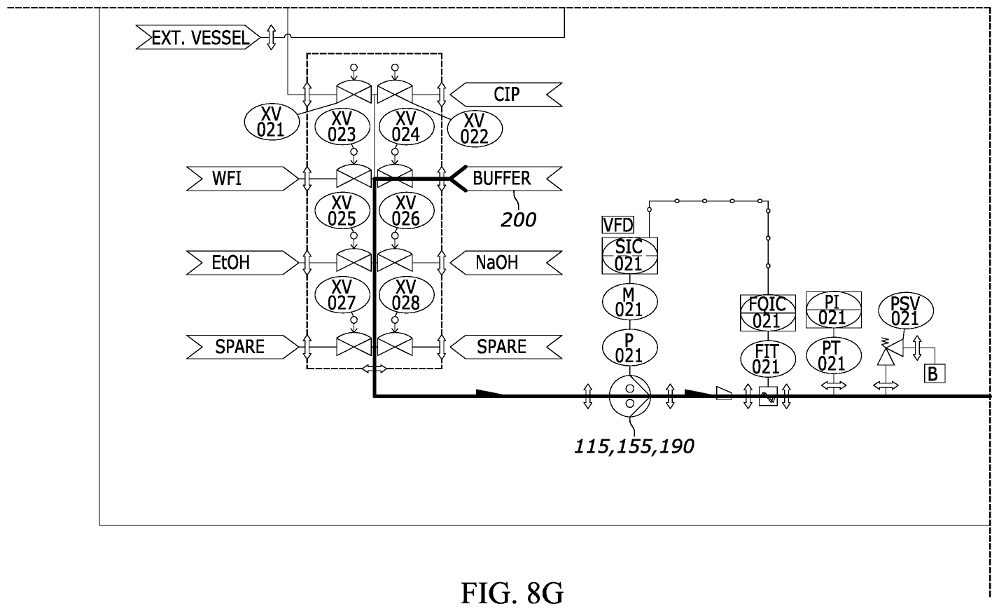

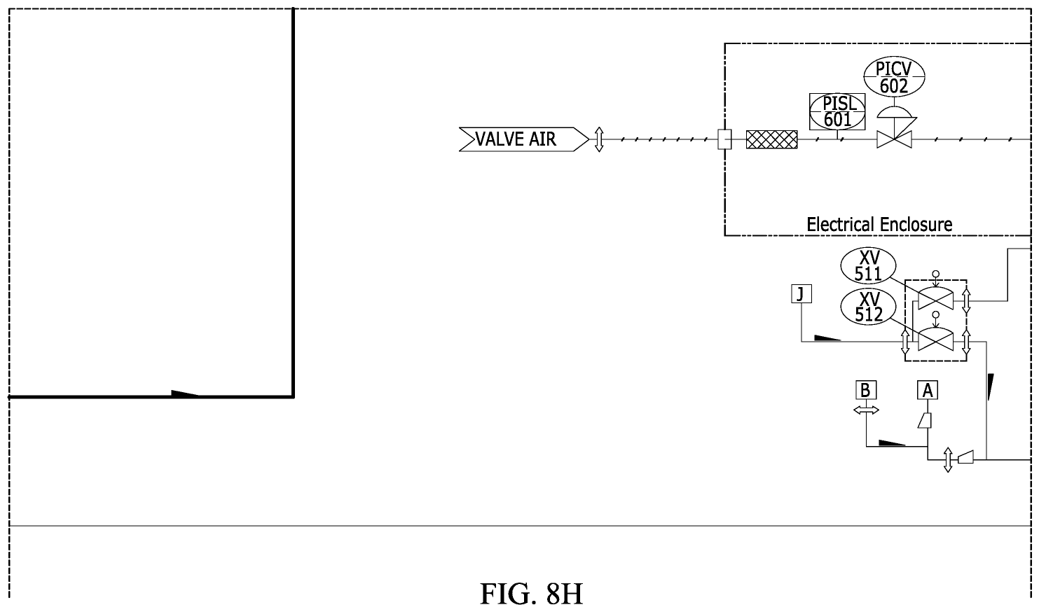

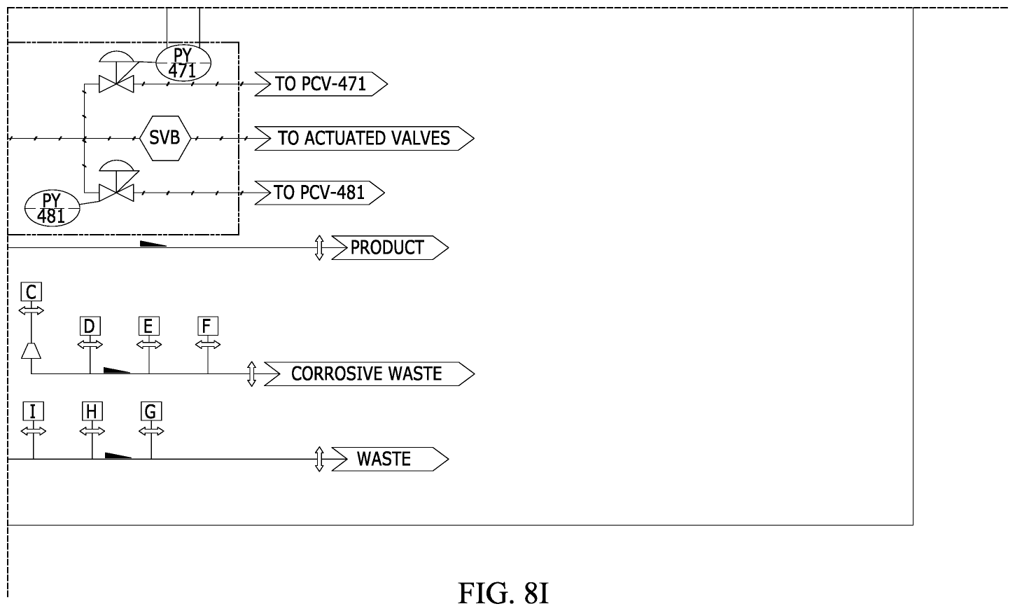

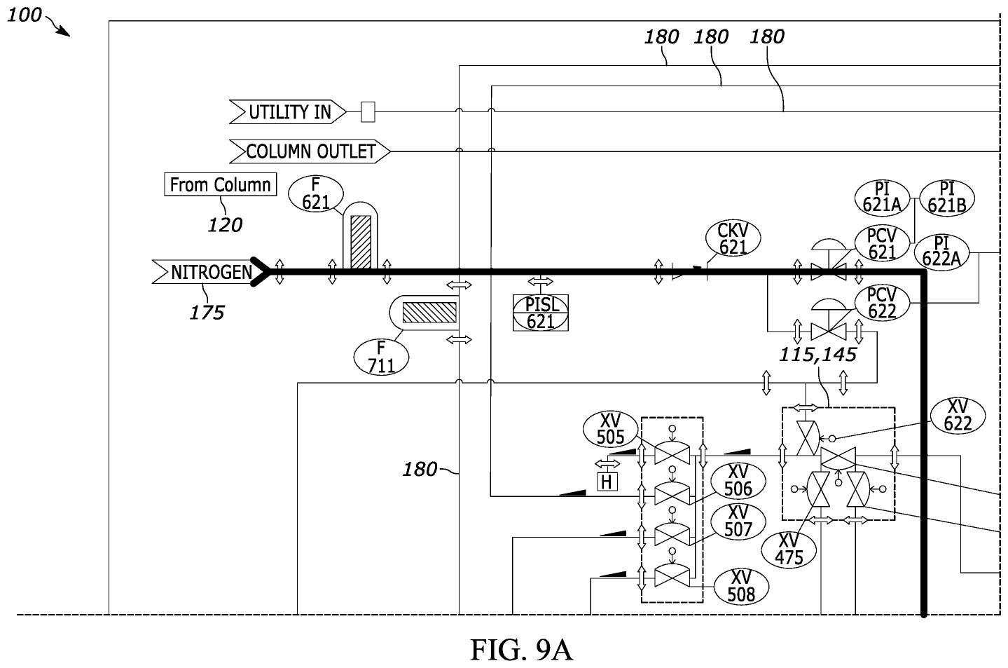

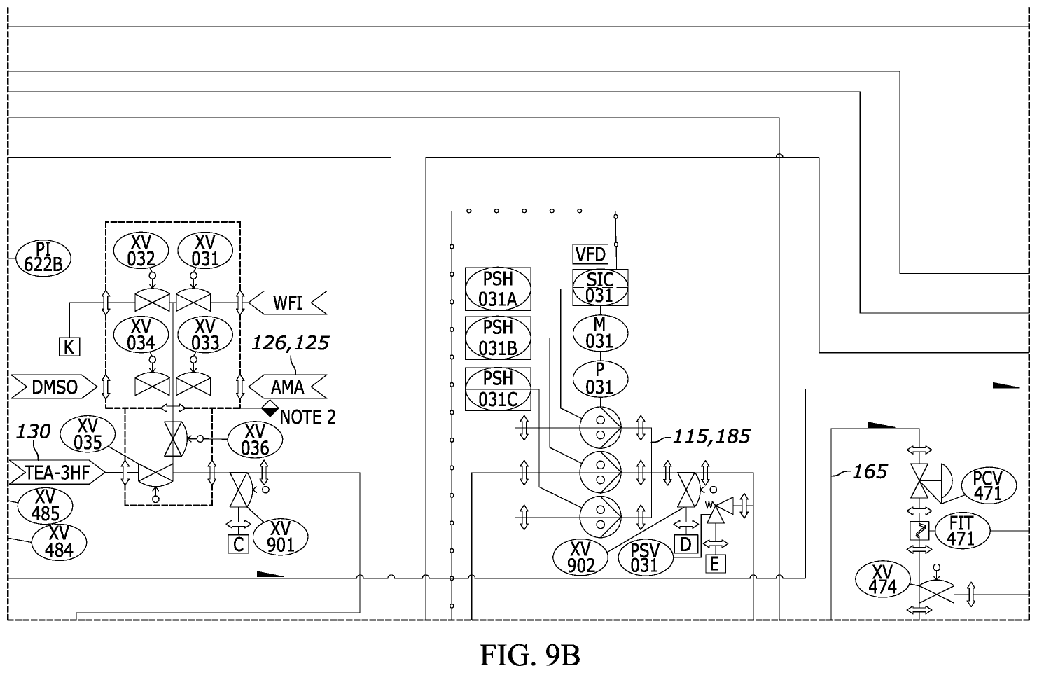

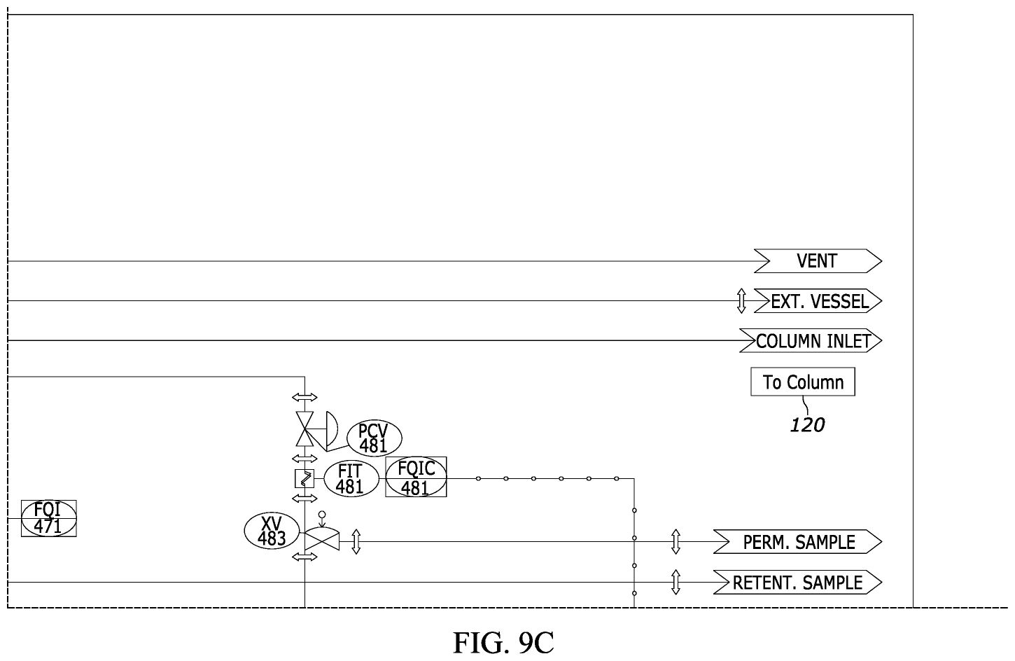

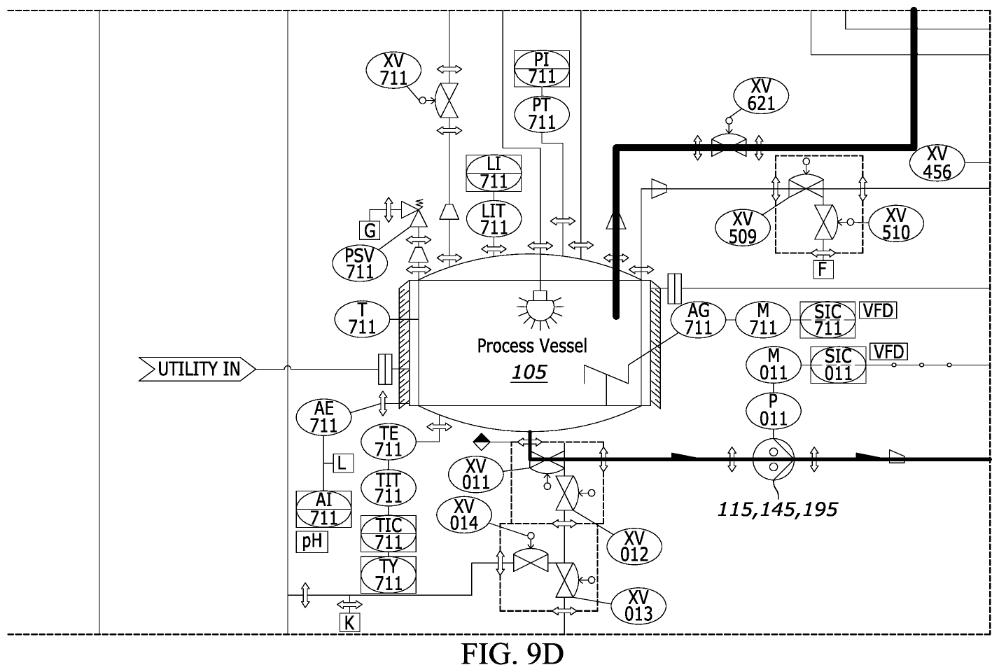

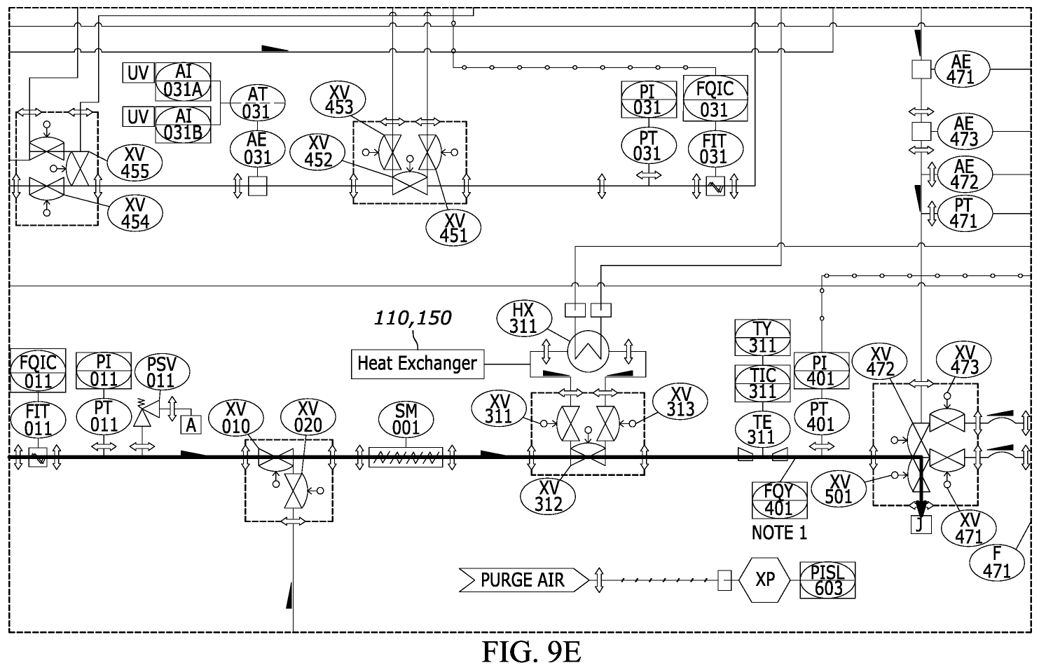

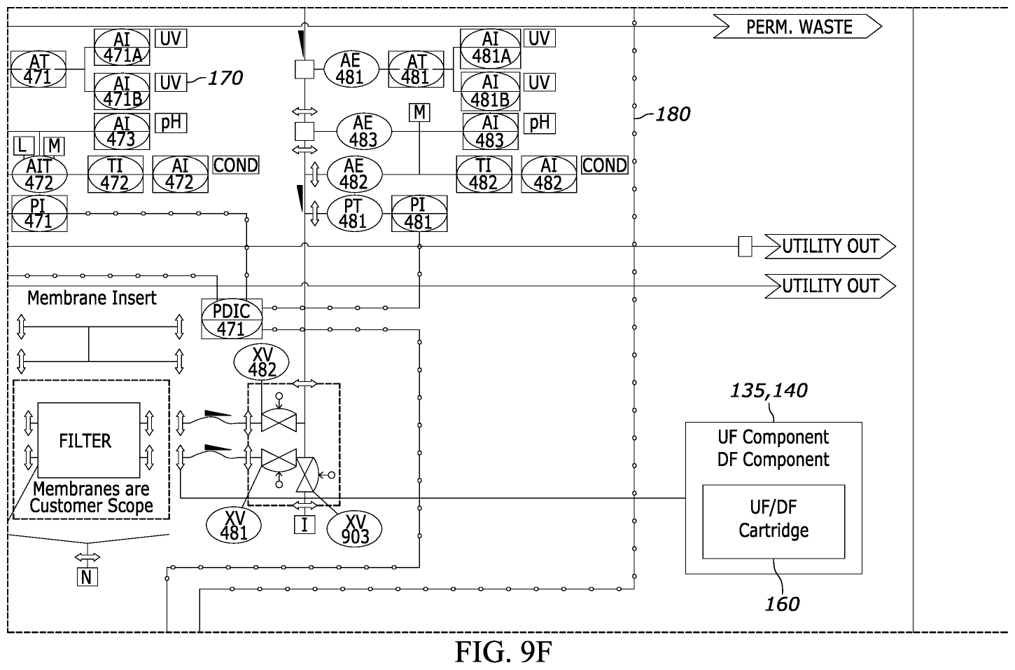

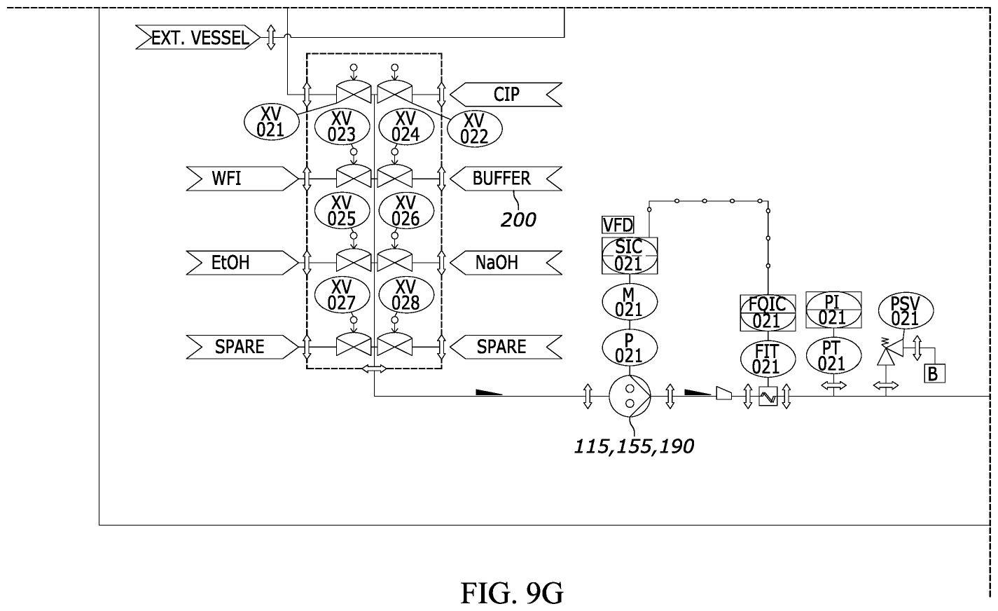

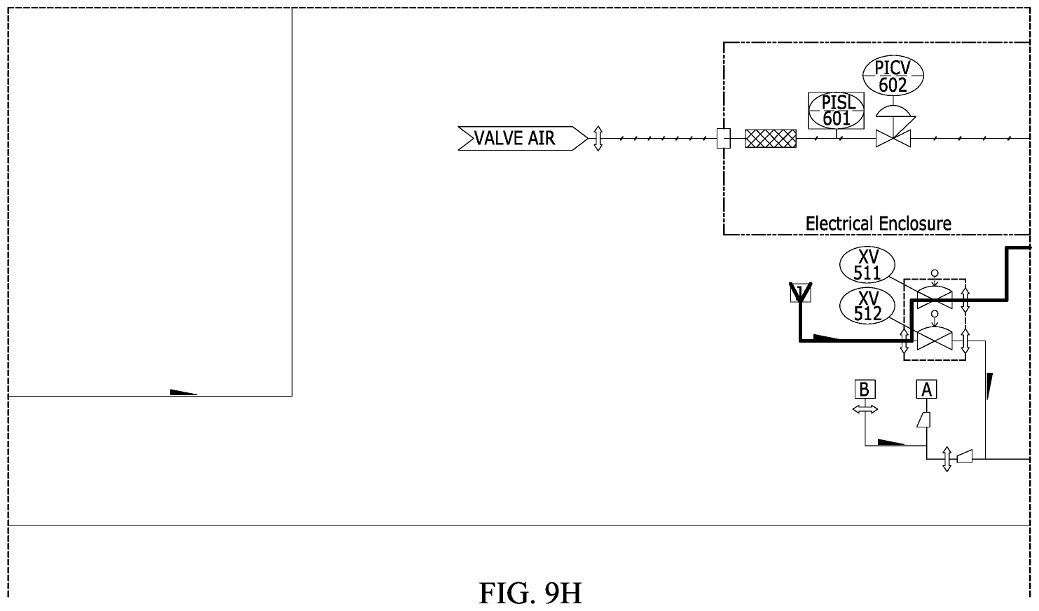

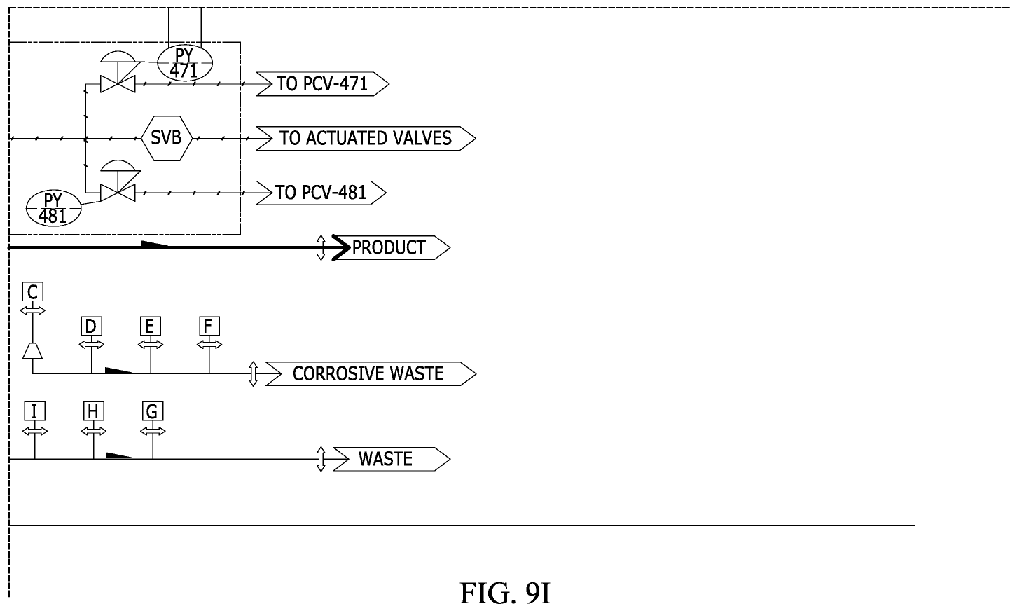

Turning now to FIGS. 8A-8I and 9A-9I, which illustrate how the diafiltration step is performed and the product is collected using the common system 100. The diafiltration step is performed using the diafiltration component 140, which in this embodiment comprises a diafiltration membrane. The diafiltration unit operation is generally accomplished by pumping a desired buffer solution from a supply thereof through the feed pump 155 and pumping the product solution from the process vessel 105 through the recirculation pump 145 with the combined streams passing through the diafiltration membrane and back into the process vessel as the permeate is directed to waste, thereby exchanging the buffer solution containing the product. Those skilled in the art will appreciate that various diafiltration strategies are possible, including passing the contents of the process vessel 105 across or through the diafiltration membrane depending on the nature of the membrane. With respect to the diafiltration membrane, the terms “across” and “through” are used interchangeably herein, and the skilled artisan will understand which term is applicable to a particular membrane.

More particularly, after the oligo product has been ultrafiltered, the oligo product undergoes a buffer exchange using diafiltration. Employing the same TFF approach with the same UF/DF membrane, water for injection (“WFI”) is introduced via inline mixing as the process is recirculated once again. The salt is removed from the process. Then a new buffer is introduced in the same manner and takes the place of the original buffer. The oligo product has been prepared for the next process step and can be charged out of the process vessel 105 using nitrogen pressure or pumped out of the tank using the recirculation pump 145. In some embodiments, the common system 100 comprises a recirculation pump 145 configured to pump the first retentate from the process vessel 105 and a buffer solution from a supply thereof through the recirculation loop 165 and to the UF/DF cartridge 160, such that the first retentate and the buffer solution are exposed to the UF/DF membrane, thereby undergoing diafiltration and producing a second permeate that is directed to waste and a second retentate comprising the oligo product that flows back into the process vessel. In some embodiments, pumping the contents of the process vessel 105 across the UF/DF cartridge 160 produces a permeate that is directed to waste, thereby reducing the total volume of the process vessel contents and/or exchanging a first solvent or buffer present in the contents of the process vessel with a second solvent or buffer. In some embodiments, the second solvent is water for injection (WFI).

It will be appreciated by those skilled in the art that the steps of the methods disclosed herein can be performed in various alternative manners and/or orders, and that the common system 100 disclosed herein can be configured to perform the steps in any desired alternative manner or order.

The common system 100 also includes a number of additional components illustrated in FIGS. 1A-9I but not specifically described herein. In this embodiment, the common system 100 includes a plurality of valves (e.g., check valves, diaphragm valves, pressure regulating valves, ball valves), pumps 115, a plurality of different sensors (e.g., flowmeters, pressure sensors, conductivity sensors, pH sensors, photometric sensors), a plurality of buffers (e.g., the quenching buffer), among other components. In some embodiments, various components that do not comprise an integral part of the common system 100 but which are associated with the system and enhance functionality, such as a plurality of other vessels (e.g., for holding the cleavage solution, the deprotection solution, the TEA-3HF, the buffers) or tanks (e.g., nitrogen tanks) are contemplated. Those skilled in the art will appreciate that the process vessel 105 can comprise such components useful in carrying out the methods disclosed herein, such as an agitator. In some embodiments, the methods disclosed herein are performed with agitation inside the process vessel 105.

The common system 100 also includes a number of additional components not specifically illustrated in FIGS. 1A-9I. While not illustrated, it will be appreciated that the common system 100 generally includes a controller in the form of a programmable logic controller that is communicatively connected (via a wired or wireless connection) to the various components of the common system to control operation of the common system. For example, the controller is communicatively connected to the process vessel 105, the recirculation pump 145, and the inline temperature control element 110 (e.g., inline heat exchanger 150) in order to automatically control the temperature of the deprotection component 130.

In various steps of the methods disclosed herein, it is desirable to control the temperature of part or all of the common system 100. In some embodiments, the temperature of the process vessel 105 is controlled. In some embodiments, the temperature of the common system 100 is controlled using the inline temperature control element 110. In some embodiments, the temperature is controlled with a heat exchanger. In some embodiments, the heat exchanger is a recirculating heat exchanger.

In various embodiments, it is desirable to perform the steps of the methods disclosed herein under an inert atmosphere, such as an argon or nitrogen atmosphere. The common systems 100 disclosed herein therefore optionally comprise a supply of the inert atmosphere, e.g., an argon or nitrogen supply 175.

In some embodiments, the systems and methods disclosed herein employ an external vessel that is not part of the common system 100. In some embodiments, one or more steps of the method are performed in the external vessel, provided that at least one step of the method is performed in the common system 100. In some embodiments, the methods disclosed herein comprise a deprotection step, followed by pumping the contents of the process vessel 105 into an external vessel and combining the contents with a quenching buffer therein. In some embodiments, the contents of the external vessel after quenching are pumped back into the process vessel 105 to perform additional steps of the method. In some embodiments, the contents of the external vessel are passed through or across the UF/DF cartridge 160 before being pumped either back into the external vessel or into the process vessel. Those skilled in the art will appreciate that any of the steps of the methods disclosed herein can be performed either in the process vessel 105 or in the external vessel, and that the contents of either vessel can be transferred to the other as needed. In some embodiments, the systems disclosed herein do not include an external vessel.

### Oligonucleotides

The common systems 100 and methods disclosed herein are useful for synthesizing or preparing oligonucleotides, also referred to herein as “oligo products”. As used herein, the term “oligonucleotide” refers to a nucleic acid. Nucleic acids, as used herein, include naturally occurring nucleic acids (e.g., DNA, RNA, and/or hybrids thereof), as well as unnaturally occurring nucleic acids. Nonlimiting examples of unnatural amino acids are those that comprise an unnatural backbone, modified backbone linkages such as phosphorothioate, unnatural or modified bases, and/or unnatural and modified termini. Exemplary nucleic acids include genomic DNA, complementary DNA (cDNA), messenger RNA (mRNA), micro RNA (miRNA), small interfering RNA (siRNA), small activating RNA (saRNA), peptide nucleic acids (PNA), antisense oligonucleotides, ribozymes, plasmids, and immune stimulating nucleic acids. Those of skill in the art will understand how to adapt the common systems 100 and methods disclosed herein for the synthesis of any desired natural or unnatural oligonucleotide.

Thus, also provided herein are oligonucleotides synthesized by the methods disclosed herein, and/or using the common systems 100 disclosed herein. In some embodiments, the oligonucleotide is a DNA oligonucleotide. In some embodiments, the oligonucleotide is an RNA oligonucleotide.

Those skilled in the art will recognize that a wide variety of modifications, alterations, and combinations can be made with respect to the above described embodiments without departing from the scope of the disclosure, and that such modifications, alterations, and combinations are to be viewed as being within the ambit of the inventive concept.

1. A common system for performing cleavage, deprotection, ultrafiltration, and diafiltration operations for producing an oligo product, comprising:
a single process vessel configured for receiving and/or performing post-synthesis processing of the oligo product;
an inline temperature control element paired with the single process vessel;
one or more pumps;
optionally an oligo column configured for solid-phase synthesis of the oligo product;
optionally a cleavage component configured to cleave the oligo product from a solid support within the oligo column;
a deprotection component configured for post-synthesis processing of the oligo product;
an ultrafiltration component configured for removing waste products of the solid-phase synthesis; and
a diafiltration component configured for performing one or more buffer exchanges,
wherein the process vessel, one or more pumps, cleavage component, deprotection component, ultrafiltration component, and diafiltration component are connected to each other via one or more conduits; and wherein
the one or more pumps are configured to fluidly connect
i) the cleavage component to the oligo column;
ii) the oligo column to the process vessel;
iii) the deprotection component to the process vessel; and
iv) the ultrafiltration and diafiltration components to the process vessel and to waste.

2. The common system of claim 1, comprising an oligo column.

3.-4. (canceled)

5. The common system of claim 1, further comprising a recirculation loop, one or more inline analytic devices, and/or a nitrogen supply.

6. The common system of claim 1, wherein the system does not include an external vessel and/or any additional pumps.

7. (canceled)

8. The common system of claim 1, wherein the inline temperature control element comprises an inline heat exchanger, and/or wherein the inline temperature control element is paired to the single process vessel with a recirculation pump to form a recirculation loop.

9.-13. (canceled)

11. The common system of claim 1, wherein the common system is certifiable for use in hazardous electrical areas.

14. The common system of claim 1, further comprising a controller communicatively connected to the process vessel, a recirculation pump, and the inline temperature control element for automatically controlling a temperature within the process vessel.

15. The common system of claim 1, wherein the one or more pumps comprise a feed pump and/or a deprotection pump.

16. (canceled)

17. The common system of claim 1, comprising a cleavage component.

18. The common system of claim 17, wherein the cleavage component comprises a cleavage solution and the cleavage solution is delivered to the oligo column via the deprotection pump.

19. The common system of claim 1, wherein the deprotection component comprises a deprotection solution and the deprotection solution is delivered to the process vessel via the deprotection pump.

20. The common system of claim 19, wherein the deprotection component further comprises a recirculation pump.

21. The common system of claim 15, wherein the deprotection pump is configured to pump the cleavage solution from a supply thereof, through the oligo column, and into the process vessel, thereby flushing the oligo product from the oligo column into the process vessel, and/or wherein the feed pump is configured to pump a quenching buffer from a supply thereof and into the process vessel, thereby combining the oligo product with the quenching buffer to form a combined product solution.

22. The common system of claim 21, wherein the deprotection pump is further configured to pump a deprotection solution from a supply thereof and into the process vessel, thereby mixing the deprotection solution into the oligo product.

23. The common system of claim 20, wherein the recirculation pump is configured to pump a product solution comprising the oligo product from the process vessel through a recirculation loop and back into the process vessel.

24.-25. (canceled)

26. The common system of claim 1, wherein the ultrafiltration component comprises an ultrafiltration membrane, and/or wherein the diafiltration component comprises a diafiltration membrane.

27. (canceled)

28. The common system of claim 1, wherein the ultrafiltration component and diafiltration component together comprise a common ultrafiltration/diafiltration cartridge (“UF/DF cartridge”) and/or an inline mixer.

29.-30. (canceled)

31. The common system of claim 21, wherein a recirculation pump is configured to pump the combined product solution and the quenching buffer from the process vessel through the recirculation loop and to a UF/DF cartridge, such that the combined product solution and the quenching buffer are exposed to a UF/DF membrane, thereby producing a first permeate that is directed to waste and a first retentate comprising the oligo product that flows back into the process vessel.

32. The common system of claim 31, wherein the recirculation pump is configured to pump the first retentate from the process vessel and a buffer solution from a supply thereof through the recirculation loop and to the UF/DF cartridge, such that the first retentate and the buffer solution are exposed to the UF/DF membrane, thereby undergoing diafiltration and producing a second permeate that is directed to waste and a second retentate comprising the oligo product that flows back into the process vessel.

33. A common system for performing cleavage, deprotection, ultrafiltration, and diafiltration operations for producing an oligo product, comprising:
an oligo column configured to configured for solid-phase synthesis of the oligo product;
a single process vessel downstream of the oligo column and configured for receiving and/or performing post-synthesis processing of the oligo product;
a supply of nitrogen, glycol, a cleavage solution, a deprotection solution, a quenching buffer, and a buffer solution;
a feed pump;
a deprotection pump;
a recirculation loop comprising a recirculation pump and an inline heat exchanger;
an ultrafiltration/diafiltration cartridge (“UF/DF cartridge”) comprising a ultrafiltration/diafiltration membrane (“UF/DF membrane”)
wherein the deprotection pump is configured to pump the cleavage solution from the supply thereof, through the oligo column, and into the process vessel, thereby flushing the oligo product from the oligo column into the process vessel;
wherein the feed pump is further configured to pump the deprotection solution from the supply thereof, through the deprotection pump, and into the process vessel, thereby mixing the deprotection solution into the oligo product;
wherein the recirculation pump is configured to pump a product solution comprising the oligo product from the process vessel through a recirculation loop and back into the process vessel;
wherein the feed pump is configured to pump the quenching buffer from the supply thereof and into the process vessel, thereby combining the oligo product with the quenching buffer to form a combined product solution;
wherein the recirculation pump is configured to pump the combined product solution and the quenching buffer from the process vessel through the recirculation loop and to the UF/DF cartridge, such that the combined product solution and the quenching buffer are exposed to the UF/DF membrane, thereby producing a first permeate that is directed to waste and a first retentate comprising the oligo product that flows back into the process vessel; and
wherein the recirculation pump is configured to pump the first retentate from the process vessel and the buffer solution from the supply thereof through the recirculation loop and to the UF/DF cartridge, such that the first retentate and the buffer solution are exposed to the UF/DF membrane, thereby undergoing diafiltration and producing a second permeate that is directed to waste and a second retentate comprising the oligo product that flows back into the process vessel.

34. A method of synthesizing an oligo product, comprising:
performing a solid-phase synthesis step using an oligo column to produce an oligo product;
performing a cleavage step to detach the oligo product from a solid support within the oligo column;
optionally performing a deprotection step to process the oligo product post solid-phase-synthesis;
performing an ultrafiltration step to separate the oligo product from waste products of the solid-phase synthesis; and
performing a diafiltration step to carry out one or more buffer exchanges,
wherein the solid-phase synthesis step, cleavage step, deprotection step, ultrafiltration step, and diafiltration step are performed using a common system.

35.-66. (canceled)
